(19) United States
(12) Patent Application Publication
(10) Pub. No.: US 2025/0161213 A1
(43) Pub. Date: May 22, 2025

(54) OIL-IN-WATER EMULSION WITH AGAR AND DIACETYL TARTARIC ACID ESTERS OF MONO-AND DI-GLYCERIDES (DATEM)

(71) Applicant: VITUX GROUP AS, Oslo (NO)

(72) Inventors: Kurt Ingar DRAGET, Oslo (NO); Morten J. DILLE, Oslo (NO); Magnus N. HATTREM, Oslo (NO)

(21) Appl. No.: 18/841,930

(22) PCT Filed: Feb. 28, 2023
(86) PCT No.: PCT/GB2023/050443

§ 371 Date: Aug. 27, 2024

(30) Foreign Application Priority Data
Feb. 28, 2022 (GB) 2202754.4

(51) Int. Cl. A61K 9/107; A61K 9/00; A61K 45/06; A61K 47/12; A61K 47/14; A61K 47/26; A61K 47/46

(52) U.S. Cl. CPC A61K 9/107; A61K 9/0053; A61K 45/06; A61K 47/12; A61K 47/14; A61K 47/26; A61K 47/46

(57) ABSTRACT

The invention relates to compositions for oral administration which are provided in the form of gelled oil-in-water emulsions, to methods for their preparation and to their use as pharmaceuticals and nutraceuticals. In particular, it relates to orally administrable, gelled oil-in-water emulsions which are self-supporting, viscoelastic solids having a gelled aqueous phase comprising a gelling agent which is agar, wherein said emulsion has a water activity in the range of about 0.4 to about 0.9 and is stabilised by a surfactant which comprises an ester of glycerol in which at least one hydroxyl group is esterified by diacetyl tartaric acid and at least one hydroxyl group is esterified by a fatty acid.

### FIELD OF THE INVENTION

The present invention generally relates to compositions for oral administration which are provided in the form of gelled oil-in-water emulsions, to methods for their preparation and to their use as delivery vehicles for pharmaceuticals and nutraceuticals. The compositions are soft, yet chewable, and can be provided in a unit dosage form which is easy to swallow.

More specifically, the invention relates to oral compositions which are acceptable to patients and consumers that wish to abstain from the consumption of animal by-products, for example those that follow a vegetarian diet or who are vegan. It also relates to oral compositions that are acceptable to pescetarians.

### BACKGROUND OF THE INVENTION

Soft chewable dosage forms are an alternative to traditional oral administration forms such as tablets, capsules, elixirs and suspensions. They are easier to swallow than tablets and capsules and are particularly suitable for the pediatric and elderly population as well as those that suffer from dysphagia. Such dosage forms are a popular choice for dietary supplements which contain vitamins and/or minerals (so-called “nutraceuticals”), and are also suitable for the delivery of active pharmaceutical ingredients (APIs). Active components (nutraceutical or pharmaceutical) may be present in the form of lipids in gelled oil-in-water emulsions, as dispersed particulates or dissolved in the oil or aqueous phase of such emulsions.

Chewable compositions in unit dose form comprising an oil-in-water emulsion in which the aqueous phase is gelled and in which the oil phase comprises a physiologically tolerable unsaturated fatty acid ester are disclosed in WO 2007/085840, for example. These compositions are suitable for the oral delivery of fish oils without any accompanying bad odor or taste. In WO 2010/041015 and WO 2012/140392, drug substances are dissolved or dispersed in the oil or aqueous phase of gelled oil-in-water emulsions in order to taste mask the drug substances.

A range of gelling agents may be used in the preparation of soft chewable dosage forms, such as the gelled oil-in-water emulsions described in WO 2007/085840, WO 2010/041015 and WO 2012/140392, however gelatin is by far the most widely used due to its availability, ease of use and its sol-gel transition temperature.

Gelatin is produced by partial hydrolysis of collagen found in the skin, bones and connective tissue of animals and is most commonly derived from pork, bovine and fish sources. The sol-gel transition temperature of a gelatin generally corresponds to the body temperature of the animal from which it is obtained. Gelatins from mammalian sources therefore have transition temperatures which are similar to human body temperature, resulting in gels which are solid at room temperature but which melt in the mouth once ingested. Gelatin-based dosage forms thus provide a pleasant ‘melt-in-the mouth’ texture or “mouthfeel”. Gelatin also provides fast and consistent dissolution kinetics of a dosage unit in the gastrointestinal tract which can be beneficial to promote rapid uptake of any active components.

Gelatin has significant surface active properties which allows it to act as an emulsifier as well as a gelling agent. This makes it a particularly good choice for use as a gelling agent to produce oil-in-water emulsions which are chewable.

Gelatin-based emulsions typically experience an “active filler effect” in which the droplets of oil interact strongly with the surrounding gel network and are generally referred to as “active fillers”. When the oil droplets are sufficiently small, this interaction between the gel network and the oil droplets increases the storage modulus of the gelled emulsion compared to an oil-free gel, i.e. the gel alone. In contrast, oil droplets which are distributed throughout a gel with little or no interaction with the gel network are known as “inactive fillers” and result in a modulus for the gelled emulsion which is lower than that of the gel alone. When oil droplets of a gelled emulsion are present as “inactive fillers”, the emulsion may not be stable over time. That can lead to destabilization of the emulsion and ‘sweating’ of oil.

Despite the many advantageous properties of gelatin for use in the production of soft chewable dosage forms, its animal origin makes it unacceptable to many patients and consumers due to their religious beliefs or dietary choices. As an animal by-product, gelatin is not acceptable to vegans for example.

Gelling agents that are not of animal origin and which have previously been proposed for use in the production of soft chewable dosage forms, such as gelled oil-in-water emulsions, include non-proteinaceous materials such as alginates, carrageenans and pectins. However, the gelling properties of these materials can be difficult to control due to the need for their complexation with metal ions, temperature change and/or pH adjustment to produce the desired ‘gel’. This is not ideal in the context of a dosage form which is to be manufactured on a commercial scale.

An alternative gelling agent which is widely used in food and other non-food applications is agar. Agar is extracted from marine red algae and comprises a polysaccharide containing galactose sub-units. It is a thermosetting polymer which gels at about 30-45° C. Agar melts at about 85-90° C. and once melted it retains a liquid state until cooled to about 40° C. Due to its large hysteresis between gelling and melting temperatures it has the potential for use in the large scale production of dosage units formed from gelled oil-in-water emulsions. However, unlike gelatin which produces soft, flexible gels that can withstand a high degree of compression before they break, agar-based gels are hard and brittle. Whereas a gelatin-based gel might withstand up to 70-90% compression before it breaks, for example, an agar-based gel will typically fragment under a deformation of as little as 20%. This severely restricts its use in the production of any dosage unit that needs to be soft and chewable and have a pleasant mouthfeel. It also prevents its use in a “push-through” blister pack.

Unlike gels based on gelatin, agar-based gels are also prone to syneresis, i.e. spontaneous release of water from the gel on ageing. Gels are a 3D network of polymers which cross-link with one another trapping water within their structure. If the polymer network is not disturbed, the water remains in place. Over time, however, the polymers which form the gel may contract or alter their conformation causing water to be expelled and shrinkage of the gel. Oozing of water out of the gel is known as “syneresis” and this must be minimised in any oral dosage unit due to the need for it to have an adequate shelf-life, i.e. it should remain stable over an extended period of time. One of the ways in which the problem of syneresis of agar gels has traditionally been addressed is by increasing the agar concentration.

However, that can lead to a harder, more solid and more brittle gel which is undesirable when seeking to provide a soft, chewable dosage form.

The use of agar as a gelling agent to produce a gelled oil-in-water emulsion presents additional challenges. Unlike gelatin, which has significant surface active properties, agar must be used with a surface active agent (“surfactant”) in order to provide an emulsion which is sufficiently stable. A wide range of food grade surfactants are known and used in the art, but the Applicant has recognized that not all of these are suitable for use in stabilizing agar-based oil-in-water emulsions that are gelled.

The type of gelled oil-in-water emulsions disclosed in WO 2007/085840, WO 2010/041015 and WO 2012/140392 represent a particular challenge when seeking to replace the gelatin with agar as a gelling agent. A pre-requisite for stabilization of any oil-in-water emulsion is that the selected surfactant has the ability to at least partly dissolve or disperse in the continuous aqueous phase (i.e. in water). In order to reduce the potential for microbial growth and thus extend shelf-life, however, gelled oil-in-water emulsions should have a low water activity, i.e. a low content of ‘free’ water. In the gelatin-based emulsions disclosed in WO 2007/085840, WO 2010/041015 and WO 2012/140392, a low water activity is achieved by the inclusion of a high content of sugar alcohols (bulking agents) in the gelled aqueous phase. As a result, these gelled emulsions differ from most conventional oil-in-water emulsions in that they have a low water activity (a_{w} below 0.8, for example, typically about 0.75). The presence of buffer salts (pH adjusting agents) in the aqueous phase also results in a relatively low pH (typically between 4 and 4.7). Together, these factors give rise to fierce competition for “free” water and the potential for loss of electrostatic charge of any surfactant that is present (due to the low pH). The Applicant has found that many conventional, low molecular weight surfactants used in food or pharmaceutical products are not capable of providing a stable emulsion under these conditions. Examples of such surfactants include LACTEM (which consists of lactic acid esters of mono- and di-glycerides) and polysorbates such as Tween 80.

There is thus a continuing need for alternative soft, yet chewable, compositions for the oral delivery of pharmaceuticals and/or nutraceuticals that are suitable for vegetarians, pescetarians and vegans. In particular, there is a need for such compositions that can provide an acceptable alternative to conventional gelatin-based oil-in-water emulsions in terms of their “chew” and mouthfeel characteristics whilst having adequate stability (i.e. shelf-life) for use as pharmaceutical and/or nutraceutical products.

### SUMMARY OF THE INVENTION

The Applicant now proposes gelled oil-in-water emulsions that are acceptable to patients and consumers that are vegetarian, pescetarian or vegan, in particular to those that follow a vegetarian or vegan diet. The emulsions employ agar as a gelling agent and are stabilised using certain low molecular weight surfactants.

These surfactants comprise an ester of glycerol in which at least one hydroxyl group is esterified by diacetyl tartaric acid and at least one hydroxyl group is esterified by a fatty acid. The Applicant has found that these particular surfactants are effective in stabilising agar-based, gelled oil-in-water emulsions having low water activity, whilst also providing a gelled emulsion having desirable rheology characteristics for the oral delivery of active agents in a soft, yet chewable, dosage form. The Applicant has also found that such surfactants can provide emulsions having a small droplet size and thus a high initial lipolysis rate following oral delivery. This can be desirable since lipolysis is a pre-requisite for intestinal uptake of lipids derived from triglycerides.

In one aspect the invention thus provides an orally administrable, gelled oil-in-water emulsion which is a self-supporting, viscoelastic solid having a gelled aqueous phase comprising a gelling agent which is agar, wherein said emulsion has a water activity in the range of about 0.4 to about 0.9 and is stabilised by a surfactant which comprises an ester of glycerol in which at least one hydroxyl group is esterified by diacetyl tartaric acid and at least one hydroxyl group is esterified by a fatty acid.

In another aspect the invention provides a method for the preparation of a gelled oil-in-water emulsion as herein described, said method comprising the steps of: forming an oil phase which comprises one or more physiologically tolerable lipids; forming an aqueous phase comprising a gelling agent which is agar; combining said oil phase and said aqueous phase to form an oil-in-water emulsion in the presence of a surfactant which comprises an ester of glycerol in which at least one hydroxyl group is esterified by diacetyl tartaric acid and at least one hydroxyl group is esterified by a fatty acid; and allowing said emulsion to gel.

In a further aspect the invention provides a gelled oil-in-water emulsion as herein described for oral use as a medicament or for oral use in therapy.

In another aspect the invention provides a gelled oil-in-water emulsion as herein described which contains at least one pharmaceutically active component for oral use in the treatment of a condition responsive to said pharmaceutically active component.

In another aspect the invention provides the use of a pharmaceutically active component in the manufacture of a medicament for oral use in the treatment of a condition responsive to said pharmaceutically active component, wherein said medicament is provided in the form of a gelled oil-in-water emulsion as herein described.

In another aspect the invention provides a method of treatment of a human or non-human animal subject (e.g. a patient) to combat a condition responsive to a pharmaceutically active agent, said method comprising the step of orally administering to said subject a pharmaceutically effective amount of said agent in the form of a gelled oil-in-water emulsion as herein described.

In another aspect the invention provides the use of a gelled oil-in-water emulsion as herein described as a nutraceutical.

### DETAILED DESCRIPTION OF THE INVENTION

### Definitions

The term “gel” refers to a form of matter that is intermediate between a solid and a liquid. The formation of a “gel” will typically involve the association or cross-linking of polymer chains to form a three-dimensional network that traps or immobilises solvent (e.g. water) within it to form a sufficiently rigid structure that is resistant to flow at ambient temperature, i.e. at a temperature below about 25° C., preferably below about 20° C. In rheological terms, a “gel” may be defined according to its storage modulus (or “elastic modulus”), G′, which represents the elastic nature (energy storage) of a material, and its loss modulus (or “viscous modulus”), G″, which represents the viscous nature (energy loss) of a material. Their ratio, tan 5 (equal to G″/G′), also referred to as the “loss tangent”, provides a measure of how much the stress and strain are out of phase with one another.

A material which is “viscoelastic” is characterised by rheological properties which resemble, in part, the rheological behaviour of a viscous fluid and, also in part, that of an elastic solid.

The gelled oil-in-water emulsions according to the invention are “self-supporting, viscoelastic solids”. This is intended to mean that they exhibit characteristics intermediate between those of a solid and a liquid, but have a dominant solid behaviour, i.e. they have rheological characteristics more similar to that of a solid than a liquid. A “solid dominant behaviour” cannot be diluted away (i.e. destroyed) by adding more solvent. In contrast, in the case of a weak (or entangled) gel lacking stable (i.e. long lived) intermolecular crosslinks, the entangled network structure of the gel can be removed by adding more solvent and can readily be destroyed even at very low shear rate/shear stress.

The gelled oil-in-water emulsions of the invention exhibit mechanical rigidity, yet in contrast to a solid they are deformable. Specifically, the gelled emulsions herein described have a storage modulus, G′, which is greater than their loss modulus, G″, (i.e. G′>G″) over a wide frequency range, for example in the frequency range from 0.001 to 10 Hz when measured at ambient temperature (i.e. at a temperature in the range of 18° C. to 25° C., e.g. at 20° C.) and 0.1% strain. Storage modulus and loss modulus may be measured using known methods, for example using a Kinexus Ultra+ Rheometer applying a C 4/40 measuring geometry. Storage modulus and loss modulus values are not expected to differ when measured using other types of rheometer within the linear viscoelastic range.

More specifically, the gelled oil-in-water emulsions herein described will have the following properties: G′>G″ over a frequency range of 0.001 to 10 Hz at 0.1% strain; and a storage modulus (G′) at ambient temperature (i.e. at a temperature in the range of 18° C. to 25° C., e.g. at 20° C.) in the range from 10 to 200,000 Pa, preferably 100 to 100,000 Pa, more preferably 500 to 50,000 Pa.

Weak gels will typically have a loss tangent, tan 5 >0.1. For strong gels, or fully developed gels, G′>>G″ and lower tan 5 values (<0.1) are observed. The gelled oil-in-water emulsions herein described would generally be considered “strong gels” at ambient temperature, i.e. at a temperature in the range of 18° C. to 25° C., e.g. at 20° C.

As used herein, the term “gelled” refers to the formation of a “gel”. The term is used herein both in relation to the physical nature of the aqueous phase of the emulsion and that of the oil-in-water emulsion. As will be understood, the oil droplets act more or less like a solid when dispersed throughout the gelled aqueous phase of the oil-in-water emulsions which are the subject of the invention. The “gelled” nature of the aqueous phase is thus also a characteristic of the oil-in-water emulsion, i.e. it can also be considered “gelled” as described herein.

Unless otherwise defined, the term “liquid” as used herein refers to a substance which flows freely and which maintains a constant volume. It includes thickened liquids and viscous liquids which flow. A “liquid” has a loss modulus (G″) which is greater than its storage modulus (G′) and a loss tangent (tan 5) which is greater than 1.

As used herein, the term “water activity” (“a_{w}”) refers to the partial vapour pressure of water in a composition at a specified temperature divided by the standard state partial vapour pressure of water at the same temperature. Water activity thus acts as a measure of the amount of free (i.e. unbound) water in a composition. It may be calculated as:

a_{w}=p/ρ₀

where p is the partial vapour pressure of water in the composition and p₀ is the vapour pressure of pure water at the same temperature.

Alternatively, water activity may be calculated as:

a_{w}=l_{w}x_{w}

where l_{w} is the activity coefficient of water and x_{w} is the mole fraction of water.

Water activity may be measured by methods known to those skilled in the art, for example by using a Rotronic Hygrolab instrument. Unless otherwise specified, water activity is measured at ambient temperature, i.e. 21° C.±2° C.

As used herein, the term “pH” is a measure of the concentration of hydrogen ions in an aqueous medium at ambient temperature, i.e. 21° C.±2° C. pH may be measured by methods known to those skilled in the art, for example using a contact electrode for pH measurement such as a calibrated Eutech Instruments pHSpear.

As used herein, the term “surfactant” refers to a surface active compound or composition which is capable of reducing the interfacial tension between two immiscible liquids, e.g. at the interface between oil and water. It may consist of a single component or it may be a mixture of components. As herein described, methods to produce the surfactant for use in the invention will typically provide a mixture of different components.

As used herein, the term “fatty acid” refers to an un-branched or branched, preferably un-branched, hydrocarbon chain having a carboxylic acid (-COOH) group at one end, conventionally denoted the a (alpha) end. The hydrocarbon chain may be saturated or (mono- or poly-) unsaturated. By convention, the numbering of the carbon atoms starts from the a-end such that the carbon atom of the carboxylic acid group is carbon atom number 1. The other end, which is usually a methyl (—CH₃) group, is conventionally denoted w (omega) such that the terminal carbon atom is the ω-carbon. Any double bonds present may be cis- or trans- in configuration. The nomenclature “ω-x” is used to signify that a double bond is located on the xth carbon-carbon bond, counting from the terminal carbon (i.e. the ω-carbon) towards the carbonyl carbon.

By “physiologically tolerable” is meant any component which is suitable for administration to a human or non-human animal body, in particular which is suitable for oral administration.

By “pharmaceutical” is meant any product intended for a medical purpose, e.g. for treating or preventing any disease, condition or disorder of a human or non-human animal body, or for preventing its recurrence, or for reducing or eliminating the symptoms of any such disease, condition or disorder. The use and production of a product as a “pharmaceutical” will be closely regulated by a government agency. It may, but need not, be prescribed by a physician. For example, it may be available “over the counter”, i.e. without a prescription.

“Treatment” or “treating” includes any therapeutic application that can benefit a human or non-human animal (e.g. a non-human mammal). Both human and veterinary treatments are within the scope of the present invention, although primarily the invention is aimed at the treatment of humans. Veterinary treatment includes the treatment of livestock and domestic animals (e.g. pets such as cats, dogs, rabbits, etc.). Treatment may be in respect of an existing disorder or it may be prophylactic.

In contrast to a pharmaceutical, a “nutraceutical” need not be the subject of regulatory approval. The term “nutraceutical” is used herein to refer to a product which is generally considered beneficial to maintain or augment the health and/or general well-being of a human or non-human animal subject. Such substances include, in particular, dietary supplements such as vitamins and minerals which are intended to augment the health of a subject (e.g. a human subject).

As will be understood, some substances may be considered both a “pharmaceutical” and a “nutraceutical”. Categorization of a substance as one or the other, or indeed both, may vary in different countries depending on local regulations relating to medicinal products. It may also be dependent on the recommended daily dosage of any given substance. Higher daily doses of certain vitamins such as vitamin D, for example, may be regulated as a pharmaceutical whereas lower daily dosages may be considered nutraceutical.

By “a pharmaceutical composition” is meant a composition in any form suitable to be used for a pharmaceutical purpose.

By a “nutraceutical composition” is meant a composition in any form suitable to be used for a nutraceutical purpose.

A “pharmaceutically effective amount” relates to an amount that will lead to the desired pharmacological and/or therapeutic effect, i.e. an amount of the agent which is effective to achieve its intended pharmaceutical purpose. While individual patient needs may vary, determination of optimal ranges for effective amounts of any active agent is within the capability of those skilled in the art.

A “nutraceutically effective amount” relates to an amount that will lead to the desired nutraceutical effect, i.e. an amount of the agent which is effective to achieve its intended nutraceutical purpose. While the individual needs of a subject may vary, determination of optimal ranges for effective amounts of any active agent is within the capability of those skilled in the art.

The term “capsule” is used herein to refer to a unitary dosage form having a casing or coating (herein referred to as the “capsule shell”) which encloses a gelled oil-in-water emulsion as herein defined.

As used herein, an “animal by-product” is intended to refer to any product derived from, isolated from, or purified from one or more parts of an animal body (e.g. bone, skin, tissue, meat, cartilage, hoof, horn, etc.). It is also intended to refer to any composition preparing by processing an animal by-product, for example derivatised, functionalised, or otherwise chemically or physically modified, animal by-products.

As used herein, an “animal by-product” is not intended to include milk, eggs, or any compound or composition that is derived from, isolated from, or purified from animal milk or animal eggs. The term “animal by-product” does not include any synthetic material, or any material obtained from any plant, fungal, bacterial or algal source.

As used herein, the term “vegetarian diet” generally refers to a diet that lacks any meat and which also lacks any animal by-product as herein defined. A “vegetarian diet” may include animal milk and animal eggs and any products derived, isolated or purified therefrom. Such a diet may also be generally known as an “ovo-lactovegetarian” diet or “lacto-ovovegetarian” diet which, in addition to food from plants, includes milk, cheese, other dairy products and eggs. A “pescetarian diet” refers to a diet in which the only source of meat is fish and seafood. A “vegan diet” refers to a diet that is totally vegetarian and which includes only food from plants (e.g. fruit, vegetables, grains, legumes, seeds and nuts). Any reference herein to a product, substance, composition or formulation which is “suitable for” a given diet means that it would be acceptable for those that follow that particular diet. The terms “vegetarian”, “pescetarian” and “vegan” are intended to refer to those who follow a vegetarian, pescetarian or vegan diet, respectively.

In a first aspect the invention provides an orally administrable, gelled oil-in-water emulsion which is a self-supporting, viscoelastic solid having a gelled aqueous phase comprising a gelling agent which is agar, wherein said emulsion has a water activity in the range of about 0.4 to about 0.9 and is stabilised by a surfactant which comprises an ester of glycerol in which at least one hydroxyl group is esterified by diacetyl tartaric acid and at least one hydroxyl group is esterified by a fatty acid.

The aqueous phase of the emulsion according to the invention comprises water and is gelled using agar as a gelling agent. The aqueous phase is also referred to herein as the “continuous phase” of the emulsion. The gelling agent may be a single type of gelling agent or it may be a mixture of different types of gelling agents. Where more than one gelling agent is used, at least one of the agents will be agar.

Agar is well known and used in the art, for example in food and other non-food applications. It is envisaged that any known type of agar may be used in the invention. As used herein, the term “agar” is intended to broadly define any product which contains a hydrocolloidal polysaccharide extracted from red seaweed, i.e. a seaweed of the family Rhodophyceae. The hydrocolloidal polysaccharide present in agar contains one or more polymers made up of subunits of galactose. Sources of agar include seaweeds belonging to the following genera: Gelidium, Gracilaria, Pterocladia and Gelidiella. Gracilaria genus is the major source of agar globally.

The nature of the agar and its properties (e.g. its gelling capacity) will vary depending on the species from which it is extracted and the extraction method used in its production, but it is envisaged that any known agar may find use in the invention. Agars obtained from Gracilaria species are typically more sulfated and therefore have a lower gelling capacity. However, their gelling properties may be enhanced by alkaline hydrolysis of the seaweed material prior to extraction. This converts the L-galactose 6-sulfate units into 3,6-anhydro-L-galactose residues which are considered to be responsible for the gelling properties of the polymer.

Alternatively, pre- and/or post-extraction, agars may be subjected to enzyme treatment to remove sulfate groups. In one embodiment, the agar for use in the invention may be one having a reduced content of sulfate groups.

Agars are linear polysaccharides made up of alternating p (1,3)- and a (1,4)-linked galactopyranose residues. A substantial part of the a-galactose residues may exist as the 3,6-anhydride derivative. The (1,3)-linked residue is the D-enantiomer, while the (1,4)-linked residue is the L-enantiomer. Natural chemical modifications of these structures by acidic groups (namely sulfate, uronate and pyruvate) as well as by non-ionic methoxy groups have been identified. Early studies suggested that agar consisted of two main fractions: a neutral fraction termed “agarose” having high gelling ability, and a charged fraction called “agaropectin” having a lower gelling ability. More recent studies have shown that agar is a complex mixture of polysaccharides ranging from essentially neutral to charged galactan molecules.

The term “agarose” refers to the neutral polysaccharide with high gelling ability made up of repeating disaccharide units of agarobiose, i.e. 4-O-(β-D-galactopyranosyl)-3,6-anhydro-a-L-galactopyranose. The polysaccharide with repeating disaccharide units of 4-O-(β-D-galactopyranosyl)-a-L-galactopyranose in which the anyhydride bridge is absent is called “agaran”. Alkaline treatment of agar removes the sulfate ester on the C6 of the 4-linked galactose units with formation of the corresponding 3,6-anhydride form. This treatment is widely used in industrial agar extraction from Gracilaria sp. to improve its gelling properties. A more detailed overview of agar can be found, for example, in Chapter 24 of the Handbook of Hydrocolloids (Sousa et al., 2021), the entire content of which is incorporated herein by reference.

Agar is globally permitted in food products by Food Safety Authorities, including the European Food Safety Authority (EFSA) as a food additive (E-406) and the Food and Drug Administration (FDA). Agar is supplied as a powder having high solubility in water, for example at least 85% (at 80° C.). Its gel strength may vary but will typically be in the range from about 700 to about 1100 g/cm² (measured in respect of a 1.5 wt. % concentration in water at 20° C.). Gel strength is measured as follows: a solution containing 1.5 wt. % of agar is prepared. The solution is left for 24 hours at 20° C. A load is then applied to the obtained gel and a maximum weight of the load that the gel can withstand for 20 seconds is measured. The value of the maximum weight per 1 cm² is the gel strength of the agar. Gel strength may be measured using a Mecmesin Electronic Texture Analyzer. Agar having a gel strength of less than 250 g/cm² is generally referred to as “low strength” agar. Due to its lower gelling strength, “low strength” agar may be less preferred for use in the invention. In one embodiment, the agar for use in the invention is not “low strength” agar as herein defined. Preferred for use in the invention is agar having a gel strength higher than 250 g/cm². For example, the agar may have a gel strength of at least 500 g/cm², at least 600 g/cm², or least 700 g/cm². In one embodiment, the agar may have a gel strength of at least 800 g/cm².

The gelling point of agar is typically in the range from 30 to 45° C. (measured at a 1.5 wt. % concentration in water at 20° C.). The melting point of agar may, for example, range from 80 to 95° C. (measured at a 1.5 wt. % concentration in water at 20° C.). In one embodiment, agar for use in the invention has a gelling point in the range from about 35 to about 45° C. (measured at a 1.5 wt. % concentration in water at 20° C.) and/or a melting point of from about 80 to 95° C., e.g. from about 85 to 92° C. (measured at a 1.5 wt. % concentration in water at 20° C.).

Agar for use in the invention can be obtained from various commercial sources.

Non-limiting examples of agars which may be used include Gelagar HDR 800 (from B. & V. srl, Italy), and Qsol™ High Solubility Agar and Qsol Agar (from Hispanagar, Spain).

The aqueous phase of the gelled oil-in-water emulsions according to the invention can comprise agar as the sole gelling agent, or it may comprise additional non-agar gelling agents. Where other gelling agents are present, these may be selected from other gelling agents known in the art. Consistent with the intended “vegetarian” or “vegan” nature of any of the products defined herein, any additional gelling agent should not be any animal by-product. For example, mammalian gelatin will not be present. Preferably, gelatin from any source (including fish gelatin) will not be present.

The gelling agent or combination of gelling agents will be present in the aqueous phase in an amount suitable to provide the desired degree of gelling as herein described. The amount will vary to some extent dependent on the precise nature of the gelling agent(s) (for example, the type of agar which is employed) and/or other components of the aqueous phase, but a suitable amount may readily be determined by those skilled in the art. Where a gelling agent other than agar is also employed, an appropriate amount may readily be selected by those skilled in the art. The amount of agar may be adjusted accordingly.

In one set of embodiments, agar may be present in the aqueous phase at a concentration of about 0.1 to about 7.5 wt. %, preferably about 0.25 to about 5 wt. %, particularly about 0.3 to about 3.5 wt. %, e.g. about 0.5 to about 3 wt. % (i.e. based on the weight of the aqueous phase). For example, it may be present at a concentration of 0.5, 1.0, 1.5, 2.0, 2.5 or 3.0 wt. % (based on the weight of the aqueous phase). The concentration of agar based on the overall weight of the composition may range from about 0.1 to about 5 wt. %, preferably from about 0.15 to about 4.5 wt. %, more preferably from about 0.2 to about 4 wt. %, e.g. from about 0.25 to about 3.5 wt. %, or from about 0.25 to about 3 wt. %. For example, it may be present at a concentration of 0.25, 0.50, 0.75, 1.0, 1.25, 1.50, 1.60, 1.75, 2.0, 2.25, 2.5, 2.75 or 3.0 wt. % (based on the overall weight of the composition).

The gelled oil-in-water emulsions herein described are stabilised by a surfactant which comprises an ester of glycerol in which at least one hydroxyl group is esterified by diacetyl tartaric acid and at least one hydroxyl group is esterified by a fatty acid.

In one embodiment, the surfactant for use in the invention is a product containing diacetyl tartaric acid esters of mono- and di-glycerides, also known as “DATEM”.

DATEM is a synthetic emulsifier made from an esterification reaction of acetic acid, tartaric acid, glycerol and fatty acids from edible sources. It can also be produced from the reaction of diacetyl tartaric anhydride with mono- and diglycerides of fatty acids that are derived from edible sources. Examples of oils that may be used in its production include sunflower oil, palm oil, rapeseed oil, and combinations thereof such as a combination of sunflower oil and palm oil.

In one embodiment, the major component of the surfactant will be diacetyl tartaric acid esters of mono- and di-glycerides. For example, the surfactant may contain diacetyl tartaric acid esters of mono- and di-glycerides in an amount of at least 50 wt. %, preferably at least 60 wt. %, at least 70 wt. %, at least 80 wt. %, at least 90 wt. %, or at least 95 wt. %.

For use in the invention, DATEM is preferably made from diacetyl tartaric anhydride. For example, it may be produced by the reaction of diacetyl tartaric anhydride with a mono-glyceride and/or a diglyceride. Reaction of the anhydride provides a product having a major fraction that carries a free carboxylic acid group (—CO₂H).

DATEM consists of mixed esters of glycerol with mono- and diacetyl tartaric acids and fatty acids from edible fats and is approved for use as a food additive by the European Food Safety Authority. It has the E-number E472e and is affirmed as GRAS. It may be supplied in the form of a high viscosity liquid (i.e. semi-liquid), or in powder form.

The major component of DATEM may be represented by the following general formula (I):

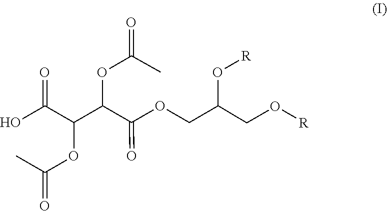

wherein each R is independently selected from H and —C(O)(CH₂)ₙCH₃ in which n is an integer from 6 to 21.

By “major component”, it is intended that the compound of formula (I) will be present in DATEM in an amount of at least 50 wt. %. In some embodiments, it may be present in an amount of at least 60 wt. %, at least 70 wt. %, at least 80 wt. %, at least 90 wt. %, or at least 95 wt. %.

Typically, stearic acid may be used as the fatty acid to produce DATEM. In the case where stearic acid is employed, the value of n will be 16 in the compound of general formula (I).

As will be understood, due to the nature of the starting materials and the method used to produce DATEM, the product consists of a mixture of components. In addition to esters of glycerol with mono- and diacetyl tartaric acids and fatty acids, it may contain any of the following: free glycerol, free glycerides, free fatty acids, free tartaric acid, free acetic acid, tartaric and acetic esters of fatty acids.

DATEM is commercially available from various suppliers. One example of DATEM that may be used in the invention is the product sold under the tradename Panodan® AB 110/C by Danisco. Panodan® AB 110/C is a diacetyl tartaric acid ester of mono-diglycerides made from edible, refined sunflower oil. It also contains the following components: citric acid (E330) (max. 50 ppm), alpha-tocopherol (E307) max. 200 ppm, and ascorbyl palmitate (E304) max. 200 ppm, and is dissolved in propylene glycol (E1520) max. 50 ppm and mono-diglyceride (E471) max. 400 ppm.

In one set of embodiments, glycerol may additionally be present in the aqueous phase of the emulsions herein described. Advantageously, glycerol may be present in an amount effective to reduce the water activity of the composition and thus reduce microbial growth. Water activity may, for example, be reduced to below about 0.8, for example in the range 0.5 to 0.8, or 0.6 to 0.75, or 0.65 to 0.75. A proportion of the water in the aqueous phase of the emulsion may, for example, be replaced by glycerol. The amount of glycerol may, for example, range from 0 to 10 wt. %, e.g. 0 to 7 wt. % based on the total weight of the composition. Typically, glycerol may be present in an amount in the range of about 6 to 7 wt. % (based on the total weight of the composition). In one embodiment, glycerol is not present.

The oil phase of the emulsion will comprise a physiologically tolerable lipid, or a mixture of different physiologically tolerable lipids. Depending on the nature of the lipid (or lipids), the oil phase itself may have nutraceutical and/or pharmaceutical properties. In some embodiments, therefore, the lipids which constitute the oil phase of the emulsion may be the nutraceutical or pharmaceutical agent.

Examples of such lipids include, for example, essential fatty acids such as those which are herein described. Alternatively, the oil phase may act as a carrier for a lipophilic pharmaceutical or nutraceutical agent. In this case, the active agent may be dissolved or dispersed in the oil phase.

A range of different lipids are known for oral use in pharmaceutical and/or nutraceutical products and any of these may be used in the oil phase of the emulsions herein described. Sources of lipids include plant oils, such as but not limited to, rapeseed oil, sunflower oil, corn oil, olive oil, sesame oil, palm kernel oil, coconut oil, nut oils (e.g. almond oil or peanut oil), algal oil and hemp oil. Fish oils and lipids obtained from fish oils are also suitable for use in certain compositions according to the invention. Compositions containing these products are acceptable to pescetarians, for example.

Lipids derived from natural products typically comprise a mixture of different lipid components. In one embodiment, the oil phase will thus comprise a mixture of different lipids. For example, it may comprise a mixture of lipids having different chain lengths and/or different degrees of saturation.

Lipids for use in the invention may be liquid, solid or semi-solid at ambient temperature (i.e. at temperatures of about 18° C. to about 25° C.). Those which are liquid at such temperatures are generally preferred. Any combination of liquid, solid and semi-solid lipids may also be used. Solid lipids having a melting point below about 100° C., preferably below about 70° C., e.g. below about 50° C. may be used in the invention. Solid lipids which may be used include butter, solid coconut fraction, cocoa butter or cocoa fat, etc. If desired, the overall melting point of the lipids which make up the oil phase may be modified by mixing different lipids, for example by mixing a solid lipid (e.g. cocoa butter) with a liquid oil. An overall melting point in the range from 45 to 50° C. may be desirable.

Lipids for use in the invention include, in particular, fatty acids and their derivatives. These include both naturally occurring fatty acids and their derivatives, as well as synthetic analogues. In one embodiment, the oil phase may comprise a mixture of different fatty acids, or fatty acid derivatives.

The hydrocarbon chain of the fatty acid or fatty acid derivative may be saturated or unsaturated, and it may be un-branched or branched. Preferably, it will be un-branched. Typically, the hydrocarbon chain will comprise from 4 to 28 carbon atoms, and generally it will have an even number of carbons. Fatty acids differ in their chain length and may be categorized as “short”, “medium”, “long”, or “very long” chain fatty acids. Those having a hydrocarbon chain of 5 or fewer carbons are referred to as “short-chain fatty acids”; those with a hydrocarbon chain of 6 to 12 carbon atoms are referred to as “medium-chain fatty acids”; those with a hydrocarbon chain of 13 to 21 carbons are referred to as “long-chain fatty acids”; and those with a hydrocarbon chain of 22 carbons or more are referred to as “very long-chain fatty acids”. Any of these may be used in the invention.

In one embodiment, the oil phase will comprise a saturated fatty acid, or a derivative of a saturated fatty acid including, but not limited to, any of the derivatives herein described. Medium-chain saturated fatty acids and their derivatives find particular use in the invention. Those having from 8 to 12, e.g. 8, 10 or 12, carbon atoms in the hydrocarbon chain are particularly preferred—i.e. caprylic acid (C8), capric acid (C10) or lauric acid (C12), and any derivatives thereof. Typically, a saturated fatty acid or derivative thereof may be used as a carrier for one or more active components in the oil phase, for example as a carrier for a pharmaceutical or nutraceutical agent.

Saturated fatty acids and their derivatives for use in the invention may be naturally occurring or they may be synthetically produced. Most typically, they will be naturally occurring and thus may be used in the form of mixtures of different fatty acids and/or different fatty acid derivatives. Sources of saturated fatty acids and their derivatives include, for example, coconut oil and palm kernel oil.

In another embodiment, the oil phase may comprise an unsaturated fatty acid or derivative thereof in which the carbon chain contains one or more carbon-carbon double bonds. The double bonds may be in the cis- or trans-configuration, or any combination thereof where more than one double bond is present. Those in which the double bonds are present in the trans-configuration are generally less preferred due to the need to reduce the consumption of so-called “trans-fats” as part of a healthy diet. Fatty acids and their derivatives having cis-configuration double bonds are thus preferred. Mono- and poly-unsaturated fatty acids and their derivatives are well known in the art. Such fatty acids typically will contain 12 to 26 carbons, more typically 16 to 22 carbons, and will have a mono- or poly-unsaturated hydrocarbon chain. They include, in particular, the polyunsaturated fatty acids (PUFAs) such as the essential fatty acids.

Oils which contain long chain, unsaturated fatty acids and their derivatives find particular use in the invention, for example in any composition which is intended for use as a nutraceutical. Particularly important essential fatty acids which may be used include the ω-3, ω-6 and ω-9 fatty acids. Examples of ω-3 fatty acids include alpha-linolenic acid (ALA), stearidonic acid (SDA), eicosatrienoic acid (ETE), eicosatetraenoic acid (ETA), eicosapentaenoic acid (EPA), docosapentaenoic acid (DPA), docosahexaenoic acid (DHA), tetracosapentaenoic acid and tetracosahexaenoic acid. Examples of ω-6 fatty acids include linoleic acid, gamma-linolenic acid, eicosadienoic acid, dihomo-gamma-linolenic acid (DGLA), arachidonic acid (AA), docosadienoic acid, adrenic acid, docosapentaenoic acid, and calendic acid. Examples of ω-9 fatty acids include oleic acid, eicosenoic acid, mead acid, erucic acid and nervonic acid.

Sources of unsaturated fatty acids and their derivatives include oils obtained from various fish, plant, algae, and microorganism sources. Particularly suitable sources are algae oils and plant oils, however fish oils may also be suitable for those that follow a pescetarian diet. These oils are all rich in ω-3, ω-6 and ω-9 fatty acids. Fish oils may, for example, be obtained from anchovies, sardines and mackerel.

Any known derivatives of the fatty acids may be used in the invention. These include, in particular, the carboxylic esters, carboxylic anhydrides, glycerides (i.e. mono-, di-, or triglycerides) and phospholipids. As used herein the term “derivatives” in the context of a fatty acid also encompasses any pharmaceutically acceptable salt of a fatty acid. Suitable salts are well known to those skilled in the art and include, but are not limited to, the lithium, sodium, potassium, ammonium, meglumine, and diethylamine salts.

Examples of carboxylic acid esters of fatty acids include compounds having a terminal —CO₂R group in which R is a straight-chained or branched alkyl group, typically a short chain alkyl, preferably a C₁₋₆ alkyl group, e.g. selected from methyl, ethyl, n-propyl, iso-propyl, n-butyl, sec-butyl and n-hexyl.

Where the fatty acid derivative is a carboxylic anhydride, it may include a terminal —CO₂COR group in which R is a straight-chained or branched alkyl group, typically a short chain alkyl, preferably a C₁₋₆ alkyl group, e.g. selected from methyl, ethyl, n-propyl, iso-propyl, n-butyl, sec-butyl and n-hexyl.

Glycerides are esters derived from glycerol and up to three fatty acids. The fatty acids present may be any of those herein described and thus they may be saturated or unsaturated, for example. In the case of di- and tri-glycerides the fatty acid components may be the same or different. For example, these may be of different chain lengths.

In one embodiment, the lipid carrier for use in the invention may comprise a medium chain triglyceride (MCT). MCTs are triglycerides with two or three medium-chain fatty acids which may be identical or different. Sources of MCTs include coconut oil and palm kernel oil, for example. The fatty acids present in MCTs are typically saturated medium chain fatty acids. MCTs from coconut oil, for example, comprise C₆₋₁₂ fatty acids, predominantly C₈ and C₁₀ fatty acids. A typical fatty acid composition of an MCT oil obtained from coconut oil may, for example, comprise: 0.1 wt. % caproic acid (C 6:0), 55 wt. % caprylic acid (C 8:0), 44.8 wt. % capric acid (C 10:0), and 0.1 wt. % lauric acid (C 12:0).

Phospholipids generally consist of a glycerol molecule linked to two fatty acids (the “tail” groups) and to a hydrophilic “head” group which consists of a phosphate group. The phosphate group may be modified by linkage to choline, ethanolamine or serine. In one embodiment, the oil phase may be constituted in whole or part by a phospholipid, for example a plant lecithin.

The amount of oil present in the compositions of the invention will be dependent on factors such as the nature of the oil, the nature and desired loading level of any pharmaceutical or nutraceutical that may be present, etc. and can be varied according to need. The oil phase may, for example, constitute from 5 to 60 wt. %, preferably from 5 to 50 wt. %, preferably from 10 to 45 wt. %, for example from 15 to 40 wt. %, from 15 to 30 wt. % or from 20 to 25 wt. % of the gelled oil-in-water emulsion.

As will be understood, in the compositions of the invention the oil provides the discontinuous phase within a continuous aqueous phase which is gelled. The oil is thus dispersed throughout the gelled aqueous phase in the form of oil droplets (also referred to herein as oil “particles”). Gelling of the aqueous phase provides a stable emulsion which prevents coalescence of the droplets of oil, for example due to the prevention of physical collisions between droplets.

The size of the oil droplets in the gelled oil-in-water emulsion is not particularly limited. However, the Applicant has found that gelled emulsions can be provided with a small oil droplet size. This may be advantageous in increasing the initial rate of lipolysis of the gelled emulsion and thus enhancing intestinal uptake of the lipids that constitute the oil phase. For example, oil droplets having a volume-based size in the range from about 100 nm to about 50 μm, preferably from about 0.25 to 20 μm, e.g. about 0.3 to 10 μm may be provided. Volume-based average oil droplet sizes may range from about 0.2 to 10 μm, preferably from about 0.4 to 5 μm, e.g. about 0.5 to 2.5 μm. “Volume-based average” as used herein refers to the volume moment mean or De Brouckere Mean Diameter (also known as the “D[4,3]” value). This reflects the size of those particles (droplets) which constitute the bulk of the sample volume and is most sensitive to the presence of large droplets in the size distribution.

An essentially homogenous size distribution of oil droplets may be desirable. The D₉₀ value indicates the size value which 90% of the oil droplets meet out of the entirety of all of the oil droplets. Do values may range from 0.75 to 50 μm, preferably 1 to 35 μm, in particular from 1.5 to 25 μm. Correspondingly, the Do and D₁₀ value, respectively, indicate the size value which 50% and 10% of the oil droplets meet out of the entirety of all of the oil droplets. Do values may range from 0.3 to 20 μm, in particular from 0.5 to 15 μm, e.g. from 0.75 to 10 μm. D₁₀ values may range from 0.1 to 10 μm, in particular from 0.2 to 5 μm, e.g. from 0.3 to 3 μm.

Lipid droplet size and size distributions can be determined using methods and apparatus conventional in the art, for example using a Malvern Mastersizer 3000 (Worcestershire, UK) connected to a Hydro MV, wet dispersion unit (Malvern, Worcestershire, UK). Analysis of the data may be performed using the manufacturer's software (Mastersizer 3000, v.0.1). Testing may be carried out by dissolving and diluting the gelled emulsion in a suitable solvent (1:100) at 50° C. Suitable solvents include Milli-Q water and a 10% (v/v) HCl solution (the latter may minimize flocculation during testing). The refractive index of water and corn oil is set to 1.33 (solvent) and 1.47 (dispersed phase), respectively, and the absorption index of the dispersed droplets set to 0.01. To avoid multiple scattering or low intensity of the scattered light, the dissolved emulsion is added to the dispersion unit (containing ˜125 mL water), until an obscuration of approximately 10% is obtained.

The size and size distribution of the oil droplets may be varied. If desired, size reduction of the oil droplets can be achieved by various different means, for example by mechanical processes or by chemical processes involving the selection of smaller lipid molecules, or indeed by a combination of these approaches. Chemical methods suitable for achieving a size reduction of the oil droplets may involve the selection of a particular type of lipid (or combination of lipids) capable of forming smaller oil droplets. Certain oils, such as MCTs for example have a tendency to produce a finer dispersion of oil droplets. Mechanical reduction involves the use of shear forces to break down larger oil droplets into smaller nano-scale particles. Smaller droplets may thus be produced by suitable adjustments to the method used to produce the emulsion, for example by varying the shear force and/or the duration of mixing of the oil and aqueous phases. The use of higher shear forces and/or longer mixing times will produce smaller droplets of oil. Suitable shear may be achieved, for example, using a conventional homogenizer such as a rotor-stator mixer, e.g. an Ultra-turrax® homogenizer. A problem often encountered in mechanical processes for the production of oil-in-water emulsions is the re-aggregation (i.e. coalescence) of the particles, but this is addressed in the invention by the use of a gelled aqueous phase which serves to stabilize the emulsion and the use of a surfactant which reduces the energy required for emulsification (by reducing interfacial tension) and which protects the droplets against re-aggregation.

The aqueous phase (i.e. continuous phase) of the gelled oil-in-water emulsion may constitute from 40 to 95 wt. %, preferably from 50 to 95 wt. %, more preferably from 55 to 90 wt. %, for example from 60 to 85 wt. %, from 70 to 85 wt. %, or from 75 to 80 wt. % of the composition.

In addition to water, the gelling agent(s) and the surfactant(s), other physiologically tolerable materials may also be present in the aqueous phase, for example, pH modifiers (e.g. buffering agents), viscosity modifiers (e.g. thickening agents, plasticizers), sweeteners, bulking agents (i.e. fillers), anti-oxidants, aromas, flavouring agents, and colouring agents. The nature and concentration of any such materials may readily be determined by those skilled in the art.

The presence of bulking agents (i.e. fillers) in the aqueous phase aids in reducing water activity and thus in reducing microbial growth. Water activity of the emulsion will be in the range from about 0.4 to about 0.9. In certain embodiments, water activity may be reduced to below about 0.8, for example in the range 0.5 to 0.8, or 0.6 to 0.75, or 0.65 to 0.75. The amount and type of bulking agents may readily be selected by those skilled in the art. Suitable examples include, but are not limited to, sugar alcohols, sugars and mixtures thereof. Suitable sugar alcohols include sorbitol, xylitol, glycerol, erythritol, maltitol, mannitol, hydrogenated starch hydrolysates (HSH), isomalt, and mixtures thereof. Sugars which may be used include any non-reducing mono- and oligosaccharides, for example trehalose, sucrose, and mixtures thereof. Bulking agents may constitute from 45 to 70 wt. %, preferably 50 to 65 wt. %, e.g. 55 to 60 wt. %, based on the aqueous phase. In some cases, the selected bulking agent(s) may also act as sweetening agents depending on their concentration. For example, the compositions according to the invention may contain xylitol, e.g. as 0.5 to 50 wt. %, preferably 1 to 40 wt. %, e.g. 15 to 40 wt. %, in order to improve taste.

Where a sweetener is included in the aqueous phase, this will typically be selected from natural sweeteners such as sucrose, fructose, glucose, reduced glucose, maltose, xylitol, maltitol, sorbitol, mannitol, lactitol, isomalt, erythritol, polyglycitol, polyglucitol, glycerol and stevia, and artificial sweeteners such as aspartame, acesulfame-K, neotame, saccharine, and sucralose. The use of non-cariogenic sweeteners is preferred.

In one embodiment, viscosity modifiers may also be provided in the aqueous phase. Suitable viscosity modifiers include other hydrocolloids such as starch, modified starch (e.g. hydroxy ethyl starch, hydroxy propyl starch), xanthan, galactomannans (e.g. guar gum and locust bean gum), gum karaya, gum tragacanth, gum arabic, gum ghatti, modified cellulose (e.g. methyl cellulose, ethyl cellulose, methylethyl cellulose, hydroxymethyl cellulose, hydroxyethyl cellulose, hydroxypropyl cellulose, hydroxyethyl ethyl cellulose, carboxymethyl cellulose, and hydroxypropylmethyl cellulose) and any combination thereof. As will be understood, a viscosity modifier may possess some surface-active properties and may additionally aid in stabilisation of the emulsion. The thickening effect of the viscosity modifier depends on the type of material (e.g. hydrocolloid) used and its concentration, the other components and the pH of the formulation, etc. but suitable amounts may readily be determined by those skilled in the art. Typical amounts of any viscosity modifier which may be present may range from 0.1 to 5 wt. % of the overall composition, preferably from 0.2 to 2.5 wt. %, for example from 0.5 to 2.0 wt. %. In one embodiment, no viscosity modifier is present.

Flavoring agents may be present in the compositions and may, for example, aid in taste masking certain lipids such as those which contain omega-3 fatty acids. Suitable flavors include, but are not limited to, citrus flavors, for example orange, lemon or lime oil.

In one embodiment, the aqueous phase of the gelled emulsion will have a low pH. For example, the pH of the aqueous phase may be less than about 7. In one set of embodiments, it may be in the range from 4 to 7, particularly 4.2 to 7, preferably 4.5 to 6, for example 4.5 to 5 or 4.5 to 4.7. pH may be adjusted by the selection of suitable pH modifiers. pH modifiers may readily be selected by those skilled in the art and include food grade acids such as citric acid. Buffering agents may also be used to adjust pH and include organic acid/base buffering systems. Suitable buffering agents are well known in the art and include, for example, sodium citrate and malic acid, etc.

Antioxidants may be present in the oil and/or aqueous phase of the compositions. Where antioxidants are present in the aqueous phase these will generally be water soluble and include, for example, ascorbic acid, citric acid and salts thereof such as sodium ascorbate. Depending on the choice of oil, these may be supplied in a form which contains an antioxidant such as vitamin E, for example. If present, the amount of any anti-oxidant(s) may be up to 3 wt. % of the overall formulation, e.g. up to 1 wt. %. In some embodiments, antioxidants may be present in the surfactant that is used in the compositions of the invention. For example, commercially available DATEM surfactants, in particular those supplied in liquid or semi-liquid form, may contain antioxidants as preserving agents. Examples of such antioxidants include citric acid, alpha-tocopherol and ascorbyl palmitate.

In addition to the lipid(s), the oil phase of the emulsion may also if desired contain physiologically tolerable lipid soluble materials, for example pharmaceutically acceptable agents, anti-oxidants (e.g. vitamin E), flavorings, and coloring agents.

In some embodiments, additional physiologically active agents may also be present in the gelled emulsions herein described. These may be provided in the aqueous and/or oil phases and may be dissolved and/or dispersed in one or both of these phases. Other actives which may be present in the oil phase include fat soluble active agents.

In one embodiment, the gelled oil-in-water emulsions according to the invention may comprise, consist essentially of, or consist of, the following components:
(a) water;
(b) a surfactant as herein defined;
(c) one or more physiologically tolerable lipids;
(d) agar;
(e) one or more bulking agents (e.g. sugar alcohols);
(f) optionally one or more pH modifiers;
(g) optionally one or more viscosity modifying agents (e.g. thickening agents or plasticisers); and
(i) optionally one or more additional physiologically active agents.

By “consisting essentially of” it is intended that the emulsions will be substantially free from (e.g. free from) other components which materially affect their properties. By “consists of” it is intended that the emulsions will be substantially free (e.g. free from) from any other components than those listed.

In one set of embodiments the compositions of the invention may be provided in the form of a dose unit. By “dose unit” it is intended that the composition will be taken orally by the subject (e.g. administered to a patient) “as received”, i.e. it will not be broken or cut before oral delivery. The weight of the dose unit will therefore be such that the composition is suitable for delivery in this way. For example, it may have an overall weight in the range from 50 to 3,000 mg, e.g. 250 to 3,000 mg or 500 to 2,500 mg, especially 100 to 2,000 mg, e.g. 750 to 2,000 mg, particularly 100 to 1,500 mg, more particularly 400 to 1,500 mg, more especially 400 to 1,000 mg. In one set of embodiments, the dose units will generally be quite large, e.g. having a mass of from 400 to 3,000 mg, e.g. 600 to 1,500 mg. The overall dose unit weight may be selected as required. For example, it may be scaled up or down dependent on the nature of the selected active components and their intended dose.

Each dose unit will typically consist of a self-supporting, gelled oil-in-water emulsion as herein described. As will be understood, in this case the dose unit will contain only the defined oil and aqueous phases, i.e. it will be free from any other components. Individual dose units may be prepared from a larger piece of the gelled emulsion which is divided, e.g. by cutting. More typically, however, each dose unit will be formed by extrusion or moulding from a liquid emulsion, or incompletely gelled emulsion, prior to gelation (i.e. above the gelling temperature of the gelling agent(s)).

Alternatively, a core of the gelled oil-in-water emulsion may be provided with a suitable coating of a physiologically tolerable coating material. Such coatings may be of the type conventional in the pharmaceutical and nutraceutical industry and may be applied by any conventional means, for example by dipping or spraying. In one set of embodiments, the gelled oil-in-water emulsions herein described may therefore be provided with a coating. For example, these may be provided within a capsule shell which dissolves in the mouth. Viewed from another aspect the invention thus provides an orally administrable capsule comprising a capsule shell enclosing a gelled oil-in-water emulsion as herein described.

In the capsules of the invention, the shell may be of any physiologically tolerable material but will typically be a sugar, a biopolymer or a synthetic or semi-synthetic polymer which is soluble or disintegrable in saliva or fluid within the gastrointestinal tract. The shell may be soft, but is preferably substantially rigid. Particularly desirably, the capsules will have the consistency of a “jelly bean”. The shell will preferably be of a material and a thickness to prevent oxidation of the contents.

The shell may comprise a sugar or cellulose, for example sorbitol. The use of sugars and cellulose as capsule shell materials is well-known in the pharmaceutical and nutraceutical fields.

The capsule shell material may thus typically be a sugar or sugar alcohol, e.g. sucrose, fructose, maltose, xylitol, maltitol or sorbitol, but may additionally contain hydrocolloid materials such as for example carageenan, alginate, pectin, cellulose, modified cellulose, starch, modified starch, gum arabic, etc. The capsule shell may contain other ingredients such as, for example, artificial sweeteners, colors, fillers, flavors, antioxidants, etc.

The capsule shell may be pre-formed such that the oil-in-water emulsion can be filled into the shell either as a liquid, or once set. Alternatively, a shell precursor (e.g. a solution) may be coated onto the set emulsion, for example using standard coating techniques. If desired the capsule may be further coated, e.g. with a wax.

Preparation of the gelled oil-in-water emulsions herein described may be carried out by emulsification of the aqueous and oil phase components. It will be understood that emulsification is carried out under conditions in which the aqueous phase is a liquid (for example a viscous liquid), i.e. prior to the formation of a gel. Emulsification will thus be carried out at a temperature above the sol-gel transition temperature of the agar gelling agent (and that of any other gelling agent that may also be present). Subsequent cooling of the emulsion below its sol-gel temperature results in the desired gelled emulsion.

Prior to emulsification, any selected active agents may be added to the oil and/or aqueous phase of the composition. This may be done, for example, by dissolving the active in the selected oil or in the aqueous phase prior to forming the emulsion. Alternatively, the selected active agent(s) may be added to a mixture of the aqueous and oil phase components prior to emulsification. During the emulsification process, the active agents will typically migrate to the oil or aqueous phase depending on their hydrophilic/lipophilic characteristics.

Emulsion formation may be effected by conventional techniques and using known equipment, for example a homogenizer based on the rotor-stator principle. The speed and duration of stirring may be adjusted as required, for example it may be varied to achieve the desired shearing force to provide the desired droplet size.

Emulsification will generally be carried out under a controlled atmosphere in order to avoid oxidative degradation of the lipid and/or any active agents. For example, emulsification may be carried out in the presence of a non-oxidising gas such as nitrogen. De-gassing to remove air bubbles may also be carried during the production process, for example prior to mixing the components of the emulsion, once the liquid emulsion has been formed, prior to packaging of the set emulsion, etc. De-gassing may be carried out using any conventional means such as the application of a vacuum, or sparging with a non-oxidising gas (e.g. nitrogen). After emulsification and gelling, the emulsion may be dried to reduce the water content. If dried, however, it will still retain a continuous gelled aqueous phase as herein described and a water content within the limits herein defined.

The gelled oil-in-water emulsions will typically be provided in dose unit form as herein described. Individual dose units may be formed by methods such as molding, extrusion or cutting. Typically, however, the dose units may be formed by filling of the liquid emulsion into molds, e.g. the individual molds of a blister pack which is then sealed. The dose units will typically be in tablet or lozenge form.

Methods for preparation of the gelled oil-in-water emulsions herein described form a further aspect of the invention. Viewed from a further aspect, the invention thus provides a method for preparing an orally administrable, gelled oil-in-water emulsion, said method comprising: forming an oil phase which comprises one or more physiologically tolerable lipids; forming an aqueous phase comprising a gelling agent which is agar; combining said oil phase and said aqueous phase to form an oil-in-water emulsion in the presence of a surfactant as herein described; and allowing said emulsion to gel. Optionally, prior to or after allowing the emulsion to gel, the emulsion may be divided into individual dose units.

The dose units are preferably individually packaged in air-tight containers, e.g. a sealed wrapper or more preferably a blister of a blister pack. In another aspect, the invention thus provides a package comprising an air-tight and light-tight compartment containing one dose unit of a composition according to the invention. By excluding both air (i.e. oxygen) and light from the packaged dose unit, long term stability of the active components is enhanced.

The packages according to the invention are preferably provided in the form of blister packs containing at least two dose units, e.g. 2 to 100, preferably 6 to 30 dose units. The blister pack will generally comprise a metal, metal/plastic laminate or plastic sheet base having molded indentations in which the dosage form is placed. The pack is normally sealed with a foil, generally a metal or a metal/plastic laminate foil, for example by applying heat and/or pressure to the areas between the indentations. The use of a metal or metal/plastic laminate to form the blister pack serves to prevent air (i.e. oxygen), light and humidity from penetrating the contents of the blister pack thus enhancing the stability of active component(s). The packages according to the invention are preferably filled under a non-oxidising gas atmosphere (e.g. nitrogen) or are flushed with such a gas before sealing.

The use of agar as a gelling agent in the compositions according to the invention provides additional advantages in relation to packaging of individual dose units in a blister pack and their removal by the end user. When an emulsion in liquid form is used to fill the indentation of the blister pack (i.e. prior to gelling), it will be in intimate contact with the inner surface of the indentation. After setting of the dose unit and sealing of the blister pack, it is important that the dose unit can easily be removed from the blister pack. The presence of gelatin, first developed as a glue, in known gelatin-based compositions can give rise to the difficulty in removing these from certain surfaces, such as those made from plastic materials, especially when a liquid emulsion containing the gelatin has been allowed to set in contact with the surface. In such cases, once set, the dose unit tends to adhere to the surface and must be torn away often causing the dose unit to fragment in the process which is not acceptable. When packaging any conventional gelatin-based dose unit, it is necessary for the internal surface of a blister pack to be coated with a suitable release agent such as a neutral oil or fat. Specially developed blister pack materials having release agents incorporated onto their internal surfaces are available but add to the cost of the packaging process. The use of a release agent also leads to a surface coating of the agent on the dose unit once it has been removed from the blister pack and this can give rise to an unpleasant feel or taste of the product.

In contrast to the use of gelatin, agar-based dose units do not adhere to conventional blister pack materials. This means that standard materials can be used, including metal/plastic laminate or a plastic film over which a plastic/metal foil laminate is heat sealed. Suitable blister trays with pre-formed cavities may, for example, be formed from laminated materials such as Tekniflex® Aclar® VA10600 (TekniPlex), Perlalux® (Perlen Packaging), Formpack® (Amcor), and Regula® (Constantia Flexibles). Such materials do not have any surface coating containing a release agent.

In one embodiment, the dose units of the invention may thus be packaged in a blister pack having an internal surface which is not coated with any release agent. Such blister packs containing a dose unit as herein described form a further aspect of the invention.

The gelled oil-in-water emulsions according to the invention find use both as pharmaceuticals, i.e. for therapeutic purposes, and as nutraceuticals to maintain or augment health and/or general well-being of a human or animal subject. For this purpose, it is intended that they are taken orally, lightly chewed in the mouth and then swallowed. It is not intended that they should remain in the mouth or need to be chewed for an extended period. Due to their soft texture, light chewing is sufficient to fragment the dosage form into smaller pieces which are easily swallowed. Based on their rheology characteristics, the gelled oil-in-water emulsions according to the invention can be expected to have a much better mouthfeel than pure aqueous agar gels. Whereas aqueous agar gels are brittle and ‘fracture’ in the mouth on chewing, the emulsions herein described have enhanced large scale deformation properties. As such, these will have a greater resistance to deformation when chewed and will be less susceptible to fracturing. Together with the small oil droplet size achieved using the surfactants herein described, this can be expected to provide a much more acceptable chewing experience for the patient or consumer.

When used as nutraceuticals, for example, the compositions herein described may be used as a supplement (e.g. as a dietary supplement) for maintaining the general health and/or well-being of a subject. Any agent known for its nutraceutical effects may be provided in the compositions and suitable agents are well known in the art. Suitable nutraceuticals include, but are not limited to, any of the following: essential fatty acids (e.g. mono and poly-unsaturated fatty acids), essential amino acids (e.g. taurine, tryptophan, tyrosine, cysteine and homocysteine), vitamins (e.g. vitamins A, B1-B12, C, D, E, K and folate), minerals (e.g. iodine, selenium, iron, zinc, calcium and magnesium), flavonoids, carotenoids (e.g. beta carotene, alpha carotene, zeaxanthin, astaxanthin, lutein, xanthophylls and lycopene), phytosterols, sapponins, probiotics, dietary fibres (e.g. insoluble fibre and beta-glucans), antioxidants (e.g. CoQ10), and plant extracts (e.g. aloe vera, evening primrose oil, garlic, ginger, ginseng, green tea, caffeine and cannabinoids). Where magnesium or calcium are present, these will generally be used in the form of their phosphate salts.

In particular, the gelled oil-in-water emulsions herein described may be used as a source of one or more essential fatty acids, such as PUFAs or their esters, e.g. omega-3, omega-6 and/or omega-9 fatty acids and their ester derivatives. Examples of omega-3 acids include a-linolenic acid (ALA), stearidonic acid (SDA), eicosatrienoic acid (ETE), eicosatetraenoic acid (ETA), eicosapentaenoic acid (EPA), docosapentaenoic acid (DPA), docosahexaenoic acid (DHA), tetracosapentaenoic acid and tetracosahexaenoic acid. Examples of omega-6 acids include linoleic acid, gamma-linolenic acid, eicosadienoic acid, dihomo-gamma-linolenic acid (DGLA), arachidonic acid (AA), docosadienoic acid, adrenic acid, docosapentaenoic acid, and calendic acid. Examples of omega-9 acids include oleic acid, eicosenoic acid, mead acid, erucic acid and nervonic acid. Omega-3 acids are especially preferred, particularly EPA and DHA.

The health benefits of essential fatty acids, in particular omega-3 fatty acids, are well known. For example, these may lower triglyceride levels and/or lower cholesterol levels. Omega-3 fatty acids are vital to everyday life and health. The beneficial effects of EPA and DHA on lowering serum triglycerides are well known. They are also known for other health benefits such as cardio-protective effects, e.g. in preventing cardiac arrhythmias, stabilising atherosclerotic plaques, reducing platelet aggregation, and reducing blood pressure. They find use therefore in treating and/or preventing vascular disease. Other benefits of omega-3 fatty acids include the prevention and/or treatment of inflammation and neurodegenerative diseases, and improved cognitive development and function.

The essential fatty acids may form part or the whole of the oil phase in the gelled emulsion, preferably at least 10% wt, more especially at least 50% wt, particularly at least 80% wt. of that phase. They may be used as single compounds or as compound mixtures, e.g. plant or marine oils. The free fatty acids, the monoacyl glycerides and diacylglycerides may be prepared by full or partial hydrolysis of triacylglycerides, for example acid, base, or enzyme-catalysed hydrolysis, e.g. using lipases such as pancreatic lipases and/or lipases which may be produced from bacteria as fermentation products. Alkyl esters of essential fatty acids may be prepared by transesterification using the appropriate alkanol or by esterification of the free fatty acid with that alkanol. Where a free fatty acid is used, this may be in acid form or salt form (e.g. wholly or partially in salt form), and preferably constitutes 5 to 75% wt, especially 10 to 35% wt. of the essential fatty acid in the oil phase. Salt forms may be preferred.

The gelled oil-in-water emulsions herein described also find use as pharmaceuticals in the treatment or prevention of a range of medical conditions which are responsive to the chosen active agent(s). As will be appreciated, the nature of such conditions will be dependent on the selected active agent(s), but can readily be determined by those skilled in the art.

Any drug substance having a desirable therapeutic and/or prophylactic effect may be used. This includes drug substances which are lipophilic, hydrophilic, or amphiphilic.

Classes of suitable drug substances include, but are not limited to, any of the following: analgesics; anti-inflammatories; anti-cancer agents; cardiovascular agents; biological agents; anti-allergy agents (e.g. antihistamines); decongestants; anti-nausea agents, drugs affecting gastrointestinal function; drugs acting on the blood and blood-forming organs; drugs affecting renal and cardiovascular function; anti-fungal agents; urological agents; hormones; antimicrobial agents, anti-epileptical agents; psycholeptical agents; antipsychotic agents; psychoanaleptical agents; anticholinesterase agents; and carotenoids.

Examples of specific drug substances which may find use in the compositions of the invention include: temazepam; diphenhydramine; zolpidem; triazolam; nitrazepam; testosterone; estradiol; progesterone; benzodiazepines; barbiturates; cyclosporine; insulin; calcitonin; dextromethorphan; pseudoephedrine; phenylpropanolamine; bromocryptine; apomorphine; selegiline; amitriptyline; dextroamphetamine; phentermine; mazindol; compazine; chlorpromazine; perphenazine; fluoxetine, buspirone; clemastine; chlorpheniramine; dexochlorpheniramine; astemizole; loratadine; paracetamol; ketoprofen; naproxen; ibuprofen; sodium acetazolamide, acetyl salicylic acid, aminophylline, amiodarone hydrochloride, ascorbic acid, atenolol, bendroflumethiazide, calcium folinate, captopril, cetrizine hydrochloride, chloramphenicol sodium succinate, chlorpheniramine maleate, chlorpromazine hydrochloride, cimetidine hydrochloride, ciprofloxacin hydrochloride, clindamycin hydrochloride, clonidine hydrochloride, codeine phosphate, cyclizine hydrochloride, cyclophosphamide, sodium dexamethasone phosphate, sodium dicloxacillin, dicyclomide hydrochloride, diltiazem hydrochloride, diphenhydramine hydrochloride, disopyramide phosphate, doxepin hydrochloride, enalapril maleate, erythromycin ethylsuccinate, flecanide acetate, fluphenazine hydrochloride, folic acid, granisteron hydrochloride, guafenesin, haloperidol lactate, hydralazin hydrochloride, hydrochloroquine sulfate, hydromorphone hydrochloride, hydroxyzine hydrochloride, sodium indomethacin, isoniazid, isoprenaline hydrochloride, ketorolac trometamol, labetalol hydrochloride, lisinopril, lithium sulfate, mesoridazine benzylate, methadone hydrochloride, methylphenidate hydrochloride, methylprednisolone sodium succinate, metorprolol tartrate, metronidazole hydrochloride, metyldopa, mexiletine hydrochloride, molidone hydrochloride, morphine sulfate, naltrexone hydrochloride, neomycin sulfate, ondanstreon hydrochloride, orciprenaline sulfate, sodium oxacillin, oxybutynin chloride, oxycodone hydrochloride, paracetamol, penicillamine, pentoxifylline, petidine hydrochloride, sodium phenobarbital, potassium phenoxymethylpenicillin, phenylephrine hydrochloride, sodium phenytoin, potassium iodide, primaquine phosphate, procainamide hydrochloride, procarbazine hydrochloride, prochlorperazine maleate, promazine hydrochloride, promethazine hydrochloride, propranolol hydrochloride, pseudoephedrine hydrochloride, pyridostigmine bromide, pyridoxine hydrochloride, ranitidine hydrochloride, salbutamol sulfate, sodium ethacrynate, sotalol hydrochloride, sumatripan succinate, terbinafine hydrochloride, terbutaline sulfate, tetracycline hydrochloride, thioridazine hydrochloride, thiothixene hydrochloride, trifluoperazine hydrochloride, triprolidine hydrochloride, sodium valproate, vancomycin hydrochloride, vancomycin hydrochloride, verapamil hydrochloride, sodium warfarin, and fenofibrate.

The quantity of drug substance per unit dose of the compositions of the invention will conveniently be in the range of 10 to 100% of the recommended daily dose for an adult or child.

Viewed from another aspect, the invention thus provides a gelled oil-in-water emulsion as herein described for use in therapy.

Viewed from still another aspect, the invention provides a gelled oil-in-water emulsion as herein described which contains at least one pharmaceutically active component for oral use in the treatment of a condition responsive to said pharmaceutically active component.

In another aspect the invention provides the use of a pharmaceutically active component in the manufacture of a medicament for oral use in the treatment of a condition responsive to said pharmaceutically active component, wherein said medicament is provided in the form of a gelled oil-in-water emulsion as herein described.

Corresponding methods of medical treatment form a further aspect of the invention. Viewed from a yet further aspect, the invention thus provides a method of treatment of a human or non-human animal subject (e.g. a patient) to combat a condition responsive to a pharmaceutically active agent, said method comprising the step of orally administering to said subject a pharmaceutically effective amount of said agent in the form of a gelled oil-in-water emulsion as herein described.

In another aspect the invention provides the use of a gelled oil-in-water emulsion as herein described as a nutraceutical. Corresponding methods of administering the gelled oil-in-water emulsion in order to achieve a nutraceutical effect also form part of the invention.

Viewed from another aspect the invention thus provides a method of administering an active agent to a human or non-human animal subject to enhance and/or maintain said subject's health or wellbeing, said method comprising the step of orally administering to said subject a nutraceutically effective amount of an active agent in the form of a gelled oil-in-water emulsion as herein described.

In another aspect the invention provides the use of a gelled oil-in-water emulsion as herein described as a nutraceutical.

When used in any of the above treatments or methods, or as nutraceutical supplements or pharmaceutical formulations, an effective amount of the active agent can readily be determined.

The effective dose level for any particular subject will depend on a variety of factors including the disorder and its severity, the identity and activity of the particular composition, the age, bodyweight, general health of the subject (e.g. patient), timing of administration, duration of treatment, other drugs being used in combination with the treatment, etc. It is well within the skill of those in the art to select the desired dose to achieve the desired therapeutic effect.

The invention will now be described further with reference to the following non-limiting Examples and the accompanying figures in which:

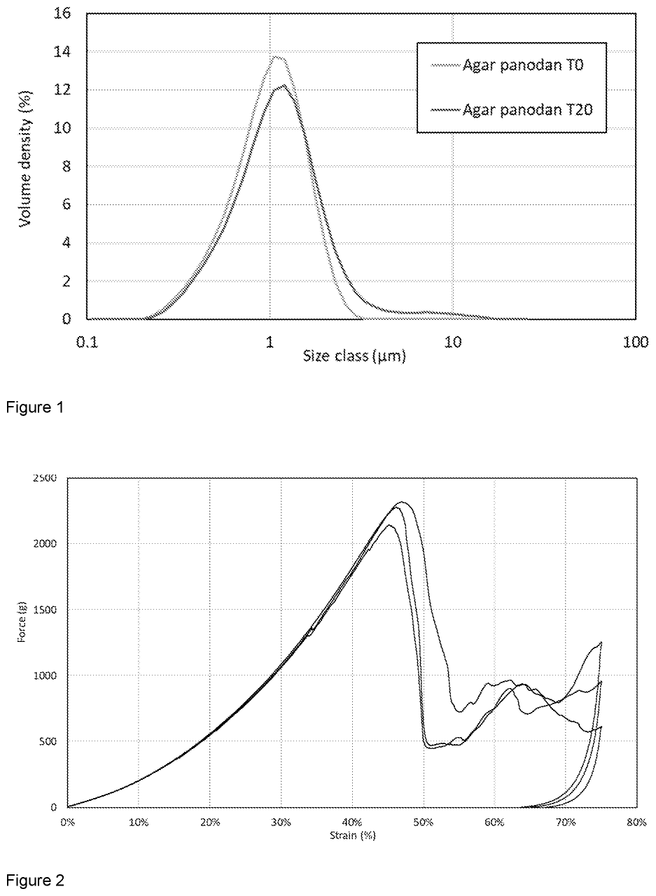

FIG. 1 shows the oil droplet size distribution for agar with 0.69 wt. % PANODAN®, measured initially and after 20 hours in a 55° C. water bath with gentle magnetic stirring.

FIG. 2 shows the hardness (force) of an agar-based gelled oil-in-water emulsion according to the invention when subjected to large scale deformation.

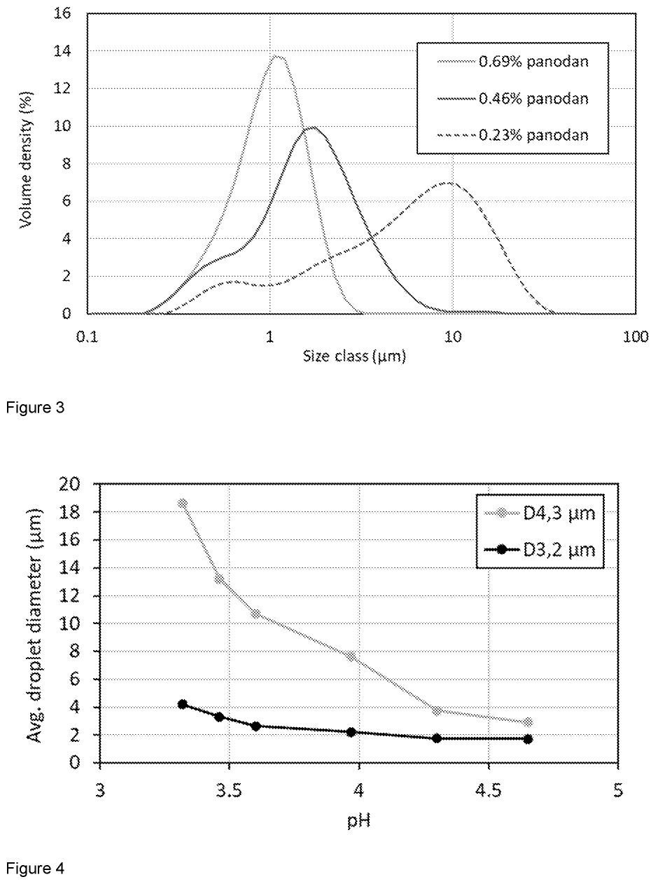

FIG. 3 shows the initial oil droplet size distribution for agar with 0.69 wt. %, 0.46 wt. % and 0.23 wt. % PANODAN® in a 55° C. water bath with gentle magnetic stirring.

FIG. 4 shows the variation in oil droplet size for agar with 0.69 wt. % PANODAN® following the addition of varying amounts of HCl.

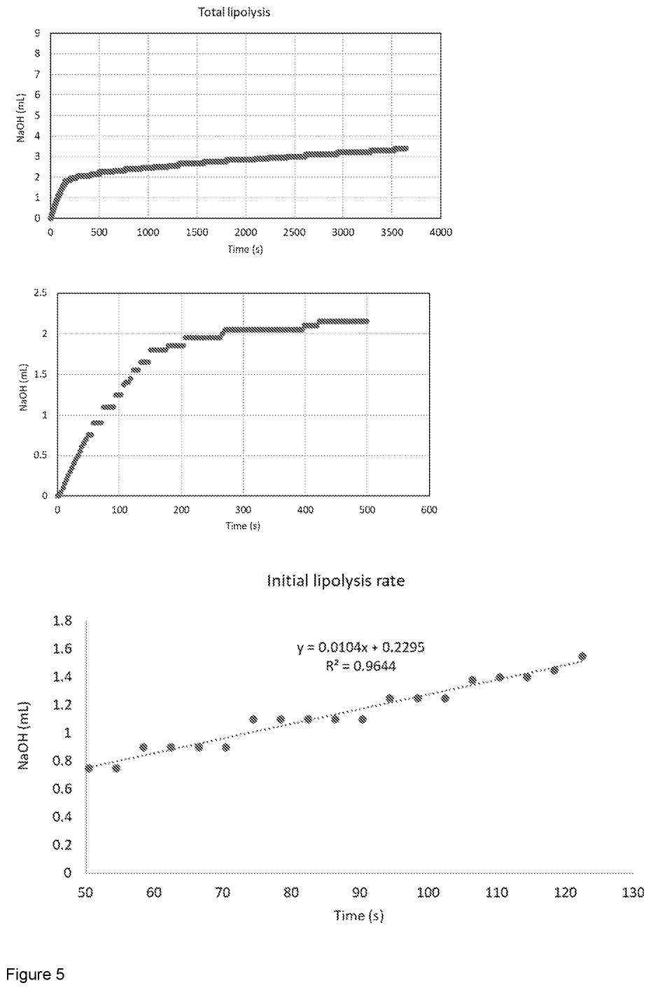

FIG. 5 shows the initial lipolysis rate of an agar-based gelled oil-in-water emulsion according to the invention.

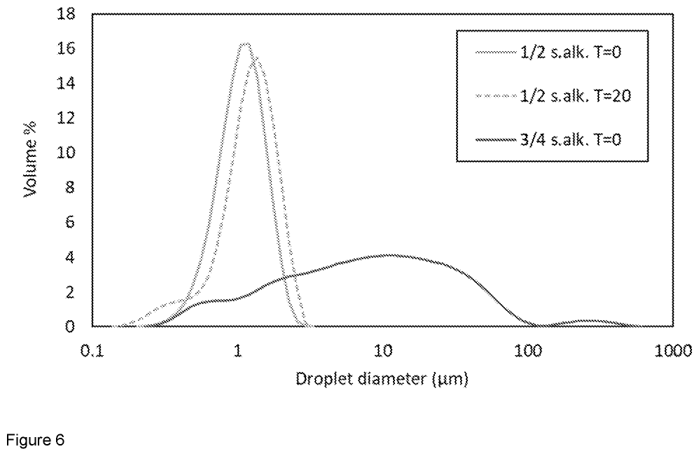

FIG. 6 shows the droplet size volume distributions for “½ s.alk” and “¾ s.alk” oil-in-water formulations with PS80 as surfactant and 25 wt. % oil.

### EXAMPLES

### Test Methods

### 1. Rheological Characterisation of Agar Emulsion Gels

1a—Small Scale Deformation

Rheological analyses on the gels were performed with a rheometer (Malvern Kinexus ultra+, Westborough, United States). The lower plate was KNX0127, 50 mm diameter curved sandblasted lower plate. The upper geometry was CP4/40 40 mm diameter 4° angle cone for gelatin emulsion gels and serrated PP40X SW1648 SS for agar gels and agar emulsion gels. Instrument calibration (zero gap) was performed prior to analysis. For the gelatin-based gels, after gel preparation approximately 2 grams of gel was placed on the lower plate, which was heated to 60° C. In the case of the agar-based gels, a liquid solution (i.e. prior to gelling) was placed on the plate and allowed to gel. The rheometer was operated in 0.1% shear strain controlled mode and the frequency was set to 1 Hz. The chosen strain was confirmed to be within the linear viscoelastic region for all samples. In order to avoid evaporation, the gelatin emulsion gel samples were covered with silicone oil (10 cS fluid, Dow Corning, UK) prior to measurement. The viscoelastic properties of the sample were obtained by using a temperature gradient of 2° C./min, with a start and end temperature at 60° C. and a holding time of 15 min at 20° C. for the gelatin emulsion gels. For agar emulsion gels, the end temperature was 90° C. and oscillation continued for 10 minutes at 90° C. The results were analyzed using rSpace for Kinexus software. The gelling and melting temperatures of the samples were estimated as the temperature at which the phase angle corresponded to 45° in the cooling and heating process, respectively. The maximum storage modulus (G′) (Pa) was determined as the highest measurement point during curing at 20° C.

1b—Large Scale Deformation

Texture properties of the gels were analysed with TA.XT plusC Texture Analyser (Stable Micro Systems Ltd., UK). Upon preparation, the gels were cast using cylindrical molds of standard dimensions (19.6 mm height, 8 mm diameter). The gels were cured at ambient temperature for 18 hours prior to analysis. Single compression analysis and the standard texture profile analysis (TPA) were performed using a 5 kg load cell. A P/35 35 mm diameter cylinder aluminum probe supplied by Stable Micro Systems Ltd. was used. For the 75% large and strain single compression, pre-test and post-test speeds were 2 mm/sec, while the test speed was 0.5 mm/sec and the trigger force was 5 grams. Strain height was measured automatically during compression. Max stress (g) and strain at failure (%) data was obtained from the fraction moment of the gels. Gradient (N/m) was calculated by the ratio of force at 2% and 3% strain. Young's modulus (N/m²) was calculated from gradient by following equation:

$$Young ’ s modulus ( N / {m}^{2} ) = \frac{Gradient ( N / m ) \times height of the gel ( m )}{Area of the gel ( {m}^{2} )}$$

Area of the gel is the contact area of the gel with the probe.

### 2. Syneresis Measurements

Syneresis measurements were based on weight loss of the gels. The gel was weighed and sealed with an air- and moisture-tight aluminum foil. Upon freezing at −20° C. and thawing at ambient temperature, and after removing excess liquid, the gel was weighed again and the difference in gel weight was normalized to percentage loss.

### 3. Water Activity Measurements

Water activity was measured with HygroPalm HC2-AW (Rotronic, Switzerland) at ambient temperature. The sample was placed into the measurement chamber and the water activity was recorded after 45 minutes.

### Example 1—Gelled Oil-In-Water Compositions and Preparation Method

### Composition:

Typical gelled oil-in-water compositions according to the invention are listed in the following table. It will be understood that any component which may be present in an amount of 0 wt. % is optional.

| Component | Wt. % |
|---|---|
| Agar (gelling agent) | 0.5-2.5 |
| Surfactant(s) | 0.25-3.5 |
| Bulking agent(s), e.g. sugar alcohol(s) | 30-60 |
| pH modifier(s) | 0-6 |
| Oil(s) | 5-60 |
| Viscosity modifier(s) | 0-5 |
| Plasticiser(s), e.g. glycerol | 0-10 |
| Anti-foaming agent(s) | 0-0.5 |
| Anti-Oxidant(s) | 0-3 |
| Sweetening agent(s) | 0-3 |
| Flavouring agent(s) | 0.05-3 |
| Colouring agent(s) | 0.001-3 |
| Pharmaceutical agent(s) | 0-10 |
| Nutraceutical agent(s) | 0-10 |
| Water | to 100 |

### Method of Preparation:

In the following method, the pH modifier is an organic acid/base buffer system consisting of trisodium citrate and malic acid, and the plasticiser (when present) is glycerol.
1. Mix agar, sugar alcohols and any sweetening agent(s) into a homogeneous powder mixture.
2. Weigh sterile water into a bottle and add the powder mixture to the water.
3. Place bottle in a water bath at 90° C. and mix with magnetic stirring for 30 minutes at 100 rpm. If glycerol is used, heat glycerol separately for 30 minutes at 60° C.
4. Reduce the temperature to 60° C. and mix the water phase with magnetic stirring for an additional 30 minutes (approx. 60 minutes in total). If glycerol is used, add the heated glycerol to the mixture with a syringe.
5. In a beaker, mix the oil together with any flavouring and/or colouring agent(s) and pre-heat to 50° C. for 30 minutes.
6. When the ingredients are completely dissolved, slowly add the surfactant and trisodium citrate to the water phase. Mix for 10 minutes and slowly add malic acid (carefully and gradually). Mix the mass for 10 more minutes.
7. If using an anti-foaming agent, add half of this agent and leave for 1 minute without stirring.
8. Weigh the bottle containing the water phase and vacuum the mass. Add the lost water (heated) and mix for 1 minute.
9. Add the oil phase into the agar mass (water phase) and homogenise the two phases for approx. 10 minutes using a high speed blender/homogeniser, such as an Ultra-Turrax T 18 at 10 000 rpm.
10. If appropriate, add the second half of the (heated) anti-foaming agent and leave for 1 minute without stirring.
11. Weigh the bottle and vacuum the mass. Add the lost water (heated) and mix for 1 minute.
12. If desired, fill the resulting emulsion into blisters of a blister pack and seal.

### Example 2—Gelled Oil-In-Water Emulsion—Typical Formulation

| Component | Wt. % |
|---|---|
| Agar (gelling agent) | 0.5-2.5 |
| Surfactant(s) | 0.25-3.5 |
| Xylitol (bulking agent) | 20-40 |
| Sorbitol (bulking agent) | 10-25 |
| Malic acid (pH modifier) | 0-2 |
| Trisodium citrate (pH modifier) | 0-4 |
| Oil | 5-60 |
| Gum Arabic | 0-2 |
| Locust Bean Gum (viscosity modifier) | 0-3 |
| Glycerol (plasticiser) | 0-10 |
| Anti-foaming agent(s) | 0-0.5 |
| Sweetening agent(s) | 0-3 |
| Flavouring agent(s) | 0.05-3 |
| Colouring agent(s) | 0.001-3 |
| Anti-oxidant(s) | 0-3 |
| Water | to 100 |

The emulsion can be prepared according to the general method in Example 1.

### Example 3—Gelled Oil-In-Water Emulsion Containing Sunflower Oil

| Ingredient | Wt. % |
|---|---|
| Agar HDR¹ | 1.60 |
| Surfactant² | 0.69 |
| Sorbitol | 14.41 |
| Xylitol | 26.85 |
| Ascorbic acid | 0.33 |
| Malic acid | 0.61 |
| Trisodium citrate | 1.87 |
| Sunflower oil | 30.00 |
| Lemon flavor | 1.10 |
| Water | 22.53 |

¹Gelagar HDR 800 (B.V. srl, Italy)
²Panodan ® AB 110/C (Danisco) - diacetyl tartaric acid ester of mono-diglycerides (DATEM) made from edible, refined sunflower oil.

The emulsion was prepared according to the general method in Example 1. The final pH of the emulsion is 4.6-4.7. pH is measured inside the gel at ambient temperature (20° C.±2° C.) using a calibrated Eutech Instruments pHSpear.

### Example 4—Multi-Vitamin Supplement

| Ingredient | Wt. % |
|---|---|
| Agar | 1.50 |
| Panodan ® AB 110/C | 0.70 |
| Sorbitol | 14.5 |
| Xylitol | 30 |
| Malic acid | 1 |
| Trisodium citrate | 2 |
| Vegetable oil¹ | 25 |
| Flavouring/colouring/sweetening | 1.50 |
| agents/ | |
| anti-foaming agent/antioxidant | |
| Water² | 24.8 |

¹Neutral carrier oil containing 10 mcg Vitamin D and 45 mcg Vitamin K
²Water phase containing 2 mcg Vitamin B12

The emulsion is prepared according to the general method in Example 1. Vitamin D and Vitamin K are added to the oil in Step 5 and Vitamin B12 is added to the water phase in Step 6.

### Example 5—Multi-Mineral Supplement

| Ingredient | Wt. % |
|---|---|
| Agar | 1.50 |
| Panodan ® AB 110/C | 0.50 |
| Sorbitol | 18.5 |
| Xylitol | 31.5 |
| Malic acid | 1 |
| Trisodium citrate | 2 |
| MCT oil | 10 |
| Flavouring/colouring/sweetening | 1.50 |
| agents/ | |
| anti-foaming agent/antioxidant | |
| Water¹ | 33.5 |

¹Water phase containing 150 mcg iodine, 40 mcg selenium, 20 mg iron and 2.5 mg zinc

The emulsion is prepared according to the general method in Example 1. The minerals are added to the water phase in Step 6.

### Example 6—Calcium Supplement

| Ingredient | Wt. % |
|---|---|
| Agar | 1.50 |
| Panodan ® AB 110/C | 0.50 |
| Sorbitol | 14.50 |
| Xylitol | 29.5 |
| Calcium Phosphate Dibasic - CaHPO₄ | 20 |
| Malic acid | 1 |
| Trisodium citrate | 2 |
| Vegetable oil¹ | 4 |
| Flavouring/colouring/sweetening | 1.50 |
| agents/anti-foaming agent/antioxidant | |
| Water | 25.5 |

¹Vegetable oil containing 400 IU Vitamin D3

The emulsion is prepared according to the general method in Example 1. The calcium phosphate is added to the water phase together with the Panodan® AB 110/C and trisodium citrate.

### Example 7—Multivitamin Supplement

| | | Amount of | |
|---|---|---|---|
| Ingredient | Wt. % | Active | |
| Water | 26.40 | | |
| Agar HDR | 1.75 | | |
| Panodan ® AB 110/C | 0.70 | | |
| Sorbitol | 15.40 | | |
| Xylitol | 31.06 | | |
| Trisodium citrate | 0.90 | | |
| Sunflower oil | 20.00 | | |
| Lemon Lime flavour | 1.20 | | |
| Paprika extract | 0.075 | | |
| Witrafrol 7420 (AF) | 0.075 | | |
| Stevia | 0.10 | | |
| Vitamin C (as Ascorbic Acid) | 1.18 | 15 | mg |
| Vitamin B3 (as Nicotinamide) | 0.56 | 8 | mg |
| Vitamin E (as D-α-Tocopherol) | 0.54 | 5 | mg |
| Vitamin B6 (as Pyridoxine HCl) | 0.064 | 0.7 | mg |
| Vitamin A (as Retinyl palmitate) | 0.029 | 200 | mcg |
| Vitamin D3 (as Cholecaliferol) | 0.016 | 5 | mcg |
| Iodine (as Potassium Iodide) | 0.011 | 30 | mcg |
| Vitamin B12 (as Cyanocobalamin) | 0.010 | 1.25 | mcg |
| Folic acid | 0.0087 | 100 | mcg |
| D-biotin | 0.0020 | 25 | mcg |

The emulsion is prepared according to the general method in Example 1. The fat soluble vitamins (E, A, D3) are mixed into the oil as in Example 4, and the water soluble vitamins (C, B3, B6, B12, folic acid, D-biotin) as well as iodine are mixed in as the CaHPO₄ is mixed in Example 6.

### Example 8—Packaging

### Blister Packs:

Prior to setting, the emulsions produced in any of Examples 1 to 7 may be filled into blister trays made from a metal/plastic laminate or a plastic film over which a plastic/metal foil laminate is heat sealed. Blister trays with pre-formed cavities may be formed from laminated materials such as Tekniflex® Aclar® VA10600 (TekniPlex), Perlalux® (Perlen Packaging), Formpack® (Amcor), and Regula® (Constantia Flexibles).

A liquid emulsion produced in any of Examples 1 to 7 is filled into blister trays using a syringe and ensuring that the cavities are filled evenly and fully. The blister trays are then flushed with nitrogen for 5-10 seconds, and sealed with a metal/plastic or metal/heat-seal lacquer cover foil by applying a flat iron set at 160° C. for 2-4 seconds. The samples are left to cure for 24 hours at room temperature, and submitted to a controlled holding chamber at 40° C. for 30 days, 65% RH. On day 5, 10, 15, 20, 25 and 30, samples were withdrawn from the controlled chamber. After 24 hours at room temperature, the blister packs are opened. The amount of residues adhering to the trays and the force required to remove the unit dose from the packs is noted on a scale from 1-9, where 1 indicates no adhesion and very little force required to remove the unit dose (“popping out”), and 9 is full adhesion to the foil and the unit dose needs to be torn from the foil. Each of the laminated materials listed above gives scores of 1, 2 or 3 (mainly 1 or 2) in each test.

### Strips:

Prior to setting, the emulsions produced in any of Examples 1 to 7 may be extruded into individual strips which, once set, are then sealed into individual plastic/metal foil laminate sachets. Alternatively, a single extruded strip, once set, may be cut into individual strips according to need prior to packaging.

### Example 9—Coated Gelled Emulsions

The set emulsions produced in any of Examples 1 to 7 may be coated with a sorbitol solution comprising sorbitol (80 wt. %), lemon flavour (0.15 wt. %), yellow colour (0.5 wt. %) and water (ad 100 wt. %). The coating solution may be cured at 99-95° C. for 4-5 hours before application. Coating is carried out by dipping or panning at 20-45° C. Several layers of coating material may be added with drying between each layer until the final composite layer is hard.

Alternatively, prior to setting, the liquid emulsion prepared in any of Examples 1 to 7 may be filled into soft capsule shells. The capsule shell material may typically be a sugar, e.g. sucrose, fructose, maltose, xylitol, maltitol or sorbitol, but may additionally contain hydrocolloid materials such as carrageenan, alginate, pectin, cellulose, modified cellulose, starch, modified starch or gum arabic. The capsule shell may contain further ingredients such as artificial sweeteners, colours, flavours and anti-oxidants.

### Example 10—Droplet Size and Size Distribution

An oil-in-water emulsion was prepared according to Example 3, then placed in a water bath at 55° C. with gentle magnetic stirring. Average droplet size was measured initially (t=0) and at 1, 2 and 20 hours after preparation.

Droplet sizes and size distributions were measured using a Malvern Mastersizer 3000 (Worcestershire, UK) connected to a Hydro MV, wet dispersion unit (Malvern, Worcestershire, UK). Analysis of the data was performed using the manufacturer's software (Mastersizer 3000, v1.0.1). Testing was carried out by diluting the emulsion in distilled water (1:100) at 70° C. The refractive index of water and oil was set to 1.33 (solvent) and 1.47 (dispersed phase), respectively, and the absorption index of the dispersed droplets set to 0.01. To avoid multiple scattering or low intensity of the scattered light, each dissolved emulsion was added to the dispersion unit (containing ˜125 mL water) until an obscuration of approximately 10% was obtained.

Droplet size distribution is shown in FIG. 1. The small increase in droplet size and tail shown at T20 came between TO and T1, and is probably not due to coalescence, but rather that some solidified agar from the side of the bottle was stirred in (particles were observed under a microscope). The lack of change in droplet size from T1 to T20 also supports that this is an artefact and not due to emulsion instability. Average droplet sizes are shown in Table 1.

TABLE 1

| Time (hours) | D [4; 3] (μm) | D [3; 2] (μm) |
|---|---|---|
| 0 | 1.07 | 0.865 |
| 1 | 1.35 | 0.943 |
| 2 | 1.34 | 0.94 |
| 20 | 1.37 | 0.947 |

### Example 11—Texture Properties

Experiments were carried out to compare the large scale deformation of a gelled agar-based emulsion prepared according to Example 3, a gelled gelatin-based emulsion, and that of a pure aqueous agar gel. The pure aqueous agar gel was prepared by mixing agar (2 wt. %) and Milli-Q-water (MQ-H₂O) at 90° C. The mixture was cooled down to ambient temperature for further characterization of the gel. The composition of the gelatin-based emulsion gel and method for its preparation are as follows:

### Composition:

| Ingredients | wt. % |
|---|---|
| Water | 21 |
| Gelatin | 8.6 |
| Xylitol | 28.4 |
| Sorbitol | 14 |
| Malic acid | 1 |
| Trisodium citrate | 2 |
| Corn oil | 25 |

### Method of Preparation:

The gelatin emulsion gel was prepared by mixing type B 160 Bloom bovine gelatin (Gelita®, Germany) with water for 30 minutes with magnetic stirring at 60° C. Sorbitol and xylitol were then added and mixed for 10 minutes after each addition. Trisodium citrate and malic acid were added and mixed for 10 minutes after each addition. Witafrol (0.06 wt. %) was added to the gel and degassed using the Diaphragm Vacuum Pump (Vacuubrand, MZ 2C) until no air bubbles were visible. The water loss was compensated after degassing. Pre-heated corn oil at 60° C. was added to the water phase and homogenized at ambient temperature at 9.8 k rpm speed for 5 minutes. The mixture was degassed again.

Tests were carried out according to the large scale deformation method described herein. The results are shown in FIG. 2 and in Table 2.

TABLE 2

| | Agar-based | | |
|---|---|---|---|
| | emulsion | Gelatin-based | |
| | 30% oil | emulsion | |
| | (Example 3) | 40% oil | Pure agar gel |
| Force at break | 2248 ± 92 | 7350 ± 150 | 670 ± 70 |
| (g) | | | |
| Strain at break | 46 ± 1 | >75% | 26 ± 1.0 |
| (%) | | | |
| Gradient | 726 ± 39 | 467 ± 36 | 586 ± 24 |
| (N/m) | | | |

The gelled agar-based emulsion according to Example 3 containing Panodan® as the surfactant provides a stronger and more flexible gel compared to the aqueous agar gel. The agar-based emulsion gel with Panodan® has an improved (longer) texture compared to the pure agar gel and approaching that of the gelatin-based emulsion gel. This will result in a more pleasant mouthfeel compared to a pure agar gel as it will not fracture dramatically when chewed.

### Example 12—Droplet Size and Size Distribution—Effect of Different Amounts of Surfactant

Gelled oil-in-water emulsions containing different amounts of Panodan® surfactant were prepared in accordance with Example 3 and tested to determine their average droplet size and size distribution. The emulsions contained 0.69 wt. %, 0.46 wt. % (⅔) and 0.23 wt. % (⅓) Panodan®, respectively. The reduction in Panodan® to 0.46 wt. % and 0.23 wt. % was compensated for by a corresponding increase in xylitol in the composition of Example 3.

Average droplet size was measured as described in Example 10 initially (t=0) and after 20 hours (T20) in a 55° C. water bath with magnetic stirring. The results are presented in FIG. 3 and Table 3.

TABLE 3

| | 0.69 wt. % | | 0.46 wt. % | | 0.23 wt. % | |
|---|---|---|---|---|---|---|
| | Panodan ® | | Panodan ® | | Panodan ® | |
| | D [4; 3] | D [3; 2] | D [4; 3] | D [3; 2] | D [4; 3] | D [3; 2] |
| | μm | μm | μm | μm | μm | μm |
| T0 | 1.07 | 0.865 | 1.91 | 1.17 | 7.47 | 2.67 |
| T20 | 1.37 | 0.947 | 2.34 | 1.27 | 9.01 | 2.98 |

The lower amount of Panodan® gave a more dispersed droplet size distribution and with larger droplets. However, the droplets were still relatively stable and no significant phase separation was observed.

### Example 13—Droplet Size and Size Distribution—Effect of Different Amounts of Oil

Oil-in-water emulsions containing different amounts of oil were prepared and tested to determine their average droplet size and size distribution. The amount of surfactant (Panodan®) in relation to the aqueous phase was kept constant (corresponding to 0.69 wt. % Panodan® in the 30 wt. % oil system). Initially 30 wt. % oil was added, then the amount of oil was gradually increased by 5 wt. % and then a further 5 wt. % with emulsification (using a large Turrax 9.8 k for 2 mins) after each addition of oil. After each emulsification, samples were taken for droplet size measurement. Average droplet size was measured as described in Example 10. The results are presented in Table 4.

TABLE 4

| Amount of oil | | | | | |
|---|---|---|---|---|---|
| (wt. %) | D [4; 3] | D [3; 2] | Dx (10) | Dx (50) | Dx (90) |
| 35 | 1.22 | 0.91 | 0.491 | 1.13 | 2.08 |
| 40 | 1.58 | 0.986 | 0.486 | 1.3 | 2.69 |
| 45 | 2.36 | 1.26 | 0.554 | 1.79 | 4.94 |
| 50 | 3.52 | 1.53 | 0.61 | 2.49 | 7.98 |
| 50 after 1 hour* | 8.07 | 2.09 | 0.729 | 5.59 | 19.2 |
| 50 after 4 hours* | 9.86 | 2.21 | 0.752 | 6.85 | 23.8 |

*measured in a 55° C. water bath with magnetic stirring

The viscosity increased significantly from 40-45 wt. % oil, and the Turrax barely managed to absorb and mix the mass at 45 wt. % oil. At 50 wt. %, the Turrax did not work and the mixture had to be emulsified by manual shaking.

It can be seen that the droplet size generally increases with increasing oil content, but even at 50 wt. % oil, the droplet size is acceptable (at least initially). At 50 wt. % oil, the droplet size increases with time although the increase from t=0 to t=1 hour is significantly greater than the increase from t=1 hour to t=4 hours. As this was homogenized by manual shaking, the apparent increase may be due to inhomogeneous emulsification which settles when placed under more quiescent conditions in the water bath.

### Example 14—Droplet Size and Size Distribution—Effect of pH

To determine the effect of pH on droplet size, an emulsion according to Example 3 was prepared with 0.69 wt. % Panodan® but without agar. The removal of agar was compensated by an increase in the content of xylitol. The emulsion was set to a temperature of 60° C. and 4M HCl was gradually added dropwise. After each addition, a small amount of the emulsion was removed for pH and droplet size measurements. pH was measured at ambient temperature (20° C.±2° C.) using a calibrated Eutech Instruments pHSpear.

The variation in droplet size based on the pH of the emulsion is shown in FIG. 4 and Table 5.

TABLE 5

| pH | D [4; 3] μm | D [3; 2] μm |
|---|---|---|
| 4.65 | 2.91 | 1.74 |
| 4.30 | 3.73 | 1.75 |
| 3.97 | 7.63 | 2.22 |
| 3.62 | 10.7 | 2.64 |
| 3.46 | 13.2 | 3.32 |
| 3.32 | 18.6 | 4.21 |

| | |
|---|---|
| 3.20 | Total phase separation |

Due to the absence of agar, the initial droplet size (at pH 4.65) is somewhat larger than the droplet sizes in the gelled agar emulsions herein described (lower viscosity and hence lower energy input). It can be seen that the droplet size increases with decreasing pH, especially from 4.30-3.97 where there is a relatively profound increase. Though not wishing to be bound by theory, this is probably due to DATEM (Panodan®) becoming partially protonated and thus gradually less effective in the low water activity gelled oil-in-water emulsion. Although the droplet size increased significantly, the droplets appeared to be relatively stable even at pH 3.32. Significant phase separation became prominent at pH 3.20.

### Example 15—Lipolysis

In vitro lipolysis of the emulsion gel according to Example 3 was performed at 37° C. Simulated intestinal fluid (SIF) was formed by mixing 450 mg bile extract porcine (Sigma, USA, lot #SLCC9272) in a beaker with 34 ml de-ionized water (dH₂O) for 30 minutes with magnetic stirring. Then, 12.5 ml salt mix (40 mM CaCl₂/600 mM NaCl/dH₂O) was added to the beaker and mixed for 10 minutes. 69.6 mg of pancreatin from porcine pancreas (Sigma, USA, lot #SLBZ5739) and lipase from porcine pancreas (Sigma, USA, lot #SLBZ7254) were dissolved in 1160 μl water in separate Eppendorf tubes through vortex mixing for 3 minutes. The gelled emulsion was pushed through a metal mesh with pore size 1.7 mm (Endecotts Ltd, UK) and 1.5 g of gel was added into the SIF whilst being mixed constantly with magnetic stirring. The pH combination electrode (A 1622M DI, SI Analytics, Germany) was placed into the beaker. The electrode was coupled to the titrator 7000-M1/20, TitroLine (SI Analytics, Germany). The pH was adjusted to 7 by manually adding droplets of 1 mM and 0.1 mM NaOH. The pH-stat method was started and 1 ml of pancreatin and lipase solutions were added to the SIF simultaneously to initiate the lipolysis reaction. The pH-stat method was set to maintain the pH at 7 by adding droplets of 0.1 mM NaOH. The total lipolysis time was 1 hour. The final concentrations of the SIF components were 9 mg/ml bile extract porcine, 10 mM CaCl₂, 150 mM NaCl, 1.2 mg/ml pancreatin and 1.2 mg/ml lipase. The final volume of the SIF was 50 ml.

The results are presented in FIG. 5 and Table 6.

TABLE 6

| | | Total NaOH | |
|---|---|---|---|
| Droplet size | | consumption | Initial lipolysis rate |
| D[4,3] | Step size | (ml) | (50-120 secs) |
| 0.61 | 0.05 | 3.4 | 0.0104 |

### Example 16—Gelled Oil-In-Water Emulsion Containing Corn Oil and Characterisation

| Ingredient | Wt. % |
|---|---|
| Agar HDR¹ | 2.00 |
| Panodan ® AB 110/C | 1.50 |
| Sorbitol | 14.00 |
| Xylitol | 28.40 |
| Malic acid | 1.00 |
| Trisodium citrate | 2.00 |
| Corn oil | 25.00 |
| Water | 26.10 |

¹Gelagar HDR 800 (B.V. srl, Italy)

The emulsion was prepared following the general method in Example 1 and according to the following protocol:

| Agar + sorbitol + xylitol | dry mix |
|---|---|
| Room temperature water | 90° C. 30 min |
| Trisodium citrate | 65° C. 10 min |
| Panodan ® | 65° C. 15 min |
| Malic acid | 65° C. 10 min |
| Witafrol (3 drops) | 70° C. degassing |
| Corn oil | 50° C. homogenization 9.8k speed |
| | for 8 mins. |

pH was measured inside the gel at ambient temperature (20° C.±2° C.) using a calibrated Eutech Instruments pHSpear. Measured pH was 4.5.

Average droplet size was measured as described in Example 10:
At T=0 hours: D [4; 3]=0.48 μm; D [3; 2]=0.44 μm

Water activity was measured as described herein. a_{w}=0.747

Average weight loss was measured as described in the syneresis tests described herein. Average weight loss: 6.96%.

Viscosity measurements were performed at 50° C. by applying the serrated PU40 SW1556 SS and sandblasted lower plate prior to the SAOS (small amplitude oscillatory shear) rheological measurements described herein. The results are as follows:

TABLE 7

| | Shear rate | Shear viscosity |
|---|---|---|
| | (s−¹) | (Pa · s) |
| t = 0 hours | 1 | 18.38 |
| | 10 | 5.123 |

Rheological analysis was carried out according to the small scale deformation test described herein.
T=0 hours: G′ max=14490 kPa.

### Example 17—Gelled Oil-In-Water Emulsion Containing Sunflower Oil

| Ingredient | Wt. % |
|---|---|
| Agar HDR¹ | 1.60 |
| Surfactant² | 0.69 |
| Sorbitol | 14.41 |
| Xylitol | 27.05 |
| Malic acid | 0.75 |
| Trisodium citrate | 1.87 |
| Sunflower oil | 30.00 |
| Lemon flavor | 1.10 |
| Water | 22.53 |

¹Gelagar HDR 800 (B.V. srl, Italy)
²Panodan ® AB 100 VEG-FS MB (Danisco) - diacetyl tartaric acid ester of mono-diglycerides (DATEM) made from edible, refined sunflower and palm oil.

The emulsion was prepared according to the general method in Example 1. Average droplet size of the emulsion at t=0 hours was measured as set out in Example 10 and the results are shown in Table 8:

TABLE 8

| Time (hours) | D [4; 3] (μm) | D [3; 2] (μm) |
|---|---|---|
| 0 | 1.25 | 0.993 |

Tests were also carried out on the gelled emulsion according to the large scale deformation method described herein. The results are set out in Table 9 with a comparison of the results for the gelled agar-based emulsion of Example 3.

TABLE 9

| | Agar-based emulsion | Agar-based emulsion |
|---|---|---|
| | 30% oil | 30% oil |
| | Panodan ® AB 110/C | Panodan ® AB 100 |
| | (Example 3) | (Example 17) |
| Force at break (g) | 2248 | 2162 |
| Strain at break (%) | 46.0 | 45.5 |

The results confirm that DATEM obtained from a different source can be used to produce a gelled oil-in-water emulsion.

### Example 18—Effect of Different Surfactants

Experiments were conducted to compare the effect of different surfactants on the stability of the emulsion. Polyoxyethylene (80) sorbitan monooleate (polysorbate 80 or “PS80”) and LACTEM (lactic acid esters of mono- and di-glycerides) were selected as examples of other food grade surfactants. Unlike DATEM, polysorbate 80 and LACTEM are both non-ionic surfactants. They are both fully neutral, i.e. they possess no electrostatic charge.

### 1. Agar Emulsion—PS80

An attempt was made to prepare oil-in-water emulsions with different amounts of sugar alcohols to determine at what point PS80 no longer functioned as an emulsifier in this system. A “standard” oil-in-water formulation (40 g) was prepared using PS80 in place of DATEM and compared to formulations in which % and % of the sugar alcohol content was replaced by water. The formulations were prepared according to the general method in Example 1. PS80 was pre-heated before being added to the remaining components of the emulsion. The formulations each contained 25 wt. % corn oil. Details of the formulations are set out in Table 10:

TABLE 10

| | | ½ sugar alcohol | ¾ sugar alcohol |
|---|---|---|---|
| Component | “Standard” | content replaced | content replaced |
| (g) | formulation | “½ s. alk” | “¾ s. alk” |
| Agar¹ | 0.81 | 0.81 | 0.81 |
| Xylitol | 11.71 | 5.86 | 8.79 |
| Sorbitol | 5.81 | 2.90 | 4.35 |
| Water | 9.83 | 18.59 | 14.21 |
| Malic acid | 0.41 | 0.41 | 0.41 |
| PS80² | 0.61 | 0.61 | 0.61 |
| Trisodium citrate | 0.81 | 0.81 | 0.81 |
| Corn oil | 10.00 | 10.00 | 10.00 |

¹Gelagar HDR 800 (B.V. srl, Italy)
²Polysorbate 80 (Sigma Aldrich)

Water activity of the formulations was measured as described herein:
“½ s.alk”: aw=0.926
“¾ s.alk”: aw=0.855

A stable emulsion could not be produced in respect of the “standard” formulation when using PS80 as the surfactant. Emulsion droplet sizes and stabilities in respect of the “% s.alk” and “% s.alk” formulations were determined as described herein. Emulsion droplet sizes and stabilities are set out in Table 11. FIG. 6 shows the droplet size volume distributions for the emulsions.

TABLE 11
Emulsion droplet sizes and stabilities

| | |
|---|---|
| T = 0 hours | T = 20 hours |

| | D [4; 3] | D [3; 2] | D [4; 3] | D [3; 2] |
|---|---|---|---|---|
| | (μm) | (μm) | (μm) | (μm) |
| “½ s. alk” | 1.12 | 0.97 | 1.27 | 1 |
| “¾ s. alk” | 25 | 3.1 | — | — |

### Observations:

½ sugar alcohols: PS80 appeared to not fully dissolve upon addition (the mixture turned turbid). It readily formed an emulsion having a very low droplet size (-1 μm) and a narrow monomodal distribution (slightly bimodal at T=20 hours). The emulsion was close to fully stable overnight at 55° C.

¾ sugar alcohols: The mixture turned turbid on addition of PS80. An emulsion seemingly formed upon homogenization, but looked yellow/translucent (indicative of the presence of large droplets). The initial droplet size was large with a wide multimodal distribution. The emulsion was highly unstable, close to full phase separation after −30 mins at 55° C.

### 2. Agar Emulsion—LACTEM

An attempt was made to prepare an oil-in-water emulsion using LACTEM as an emulsifier. The components set out below were combined according to the general method in Example 1:

| Ingredient | Wt. % |
|---|---|
| Agar HDR¹ | 1.63 |
| LACTEM² | 1.22 |
| Sorbitol | 11.61 |
| Xylitol | 23.43 |
| Malic acid | 0.81 |
| Trisodium citrate | 1.63 |
| Corn oil | 40.00 |
| Water | 19.66 |

¹Gelagar HDR 800 (B.V. srl, Italy)
²Grinsted Lactem B 30 Veg MB (Danisco)

### Observations:

The components were totally incompatible. Immediate phase separation was observed.

The results show that surfactants that are fully esterified (i.e. having no free acid functions such as carboxylic acid groups) and surfactants carrying no electrostatic charge do not function to effectively stabilise an agar-based oil-in-water emulsion having a low water activity due to their low solubility in the continuous aqueous phase. DATEM (diacetyl tartaric acid ester with mono- and di-glycerides from sunflower oil) always carries one charge. The results presented herein confirm that DATEM provides stable emulsions well below pH 3.5 and that these emulsions contain small oil droplets.

1. An orally administrable, gelled oil-in-water emulsion which is a self-supporting, viscoelastic solid having a gelled aqueous phase comprising a gelling agent which is agar, wherein said emulsion has a water activity in the range of about 0.4 to about 0.9 and is stabilised by a surfactant which comprises an ester of glycerol in which at least one hydroxyl group is esterified by diacetyl tartaric acid and at least one hydroxyl group is esterified by a fatty acid.

2. An orally administrable, gelled oil-in-water emulsion as claimed in claim 1, wherein the gelled aqueous phase of the emulsion has a pH of less than 7.

3. An orally administrable, gelled oil-in-water emulsion as claimed in claim 1 or claim 2, wherein said surfactant comprises diacetyl tartaric acid esters of mono- and di-glycerides (DATEM).

4. An orally administrable, gelled oil-in-water emulsion as claimed in claim 3, wherein said surfactant is obtained by the reaction of diacetyl tartaric anhydride with mono- and diglycerides of fatty acids obtained from an edible oil.

5. An orally administrable, gelled oil-in-water emulsion as claimed in claim 4, wherein said edible oil is sunflower oil or palm oil, or a combination thereof.

6. An orally administrable, gelled oil-in-water emulsion as claimed in claim 1, wherein said surfactant has the E-number E472e.

7. An orally administrable, gelled oil-in-water emulsion as claimed in claim 1, wherein said surfactant comprises a compound having the following general formula: 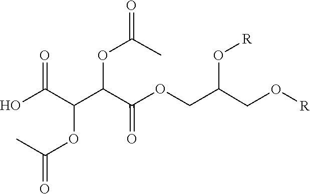
wherein each R is independently selected from H and —C(O)(CH₂)ₙCH₃ in which n is an integer from 6 to 21, preferably wherein n is 16.

8. An orally administrable, gelled oil-in-water emulsion as claimed in any one of the preceding claims, wherein said emulsion has a water activity in the range from 0.5 to 0.8.

9. An orally administrable, gelled oil-in-water emulsion as claimed in any one of the preceding claims, wherein said emulsion has a gelled aqueous phase having a pH of 4.2 to 7.

10. An orally administrable, gelled oil-in-water emulsion as claimed in any one of the preceding claims which contains agar at a concentration from about 0.1 to about 5 wt. % based on the total weight of the emulsion.

11. An orally administrable, gelled oil-in-water emulsion as claimed in any one of the preceding claims, wherein said aqueous phase constitutes from 40 to 95 wt. %, preferably from 50 to 95 wt. %, more preferably from 55 to 90 wt. %, for example from 60 to 85 wt. %, from 70 to 85 wt. %, or from 75 to 80 wt. % of the emulsion.

12. An orally administrable, gelled oil-in-water emulsion as claimed in any one of the preceding claims, wherein the aqueous phase further comprises one or more bulking agents, for example sugar alcohols and/or sugars.

13. An orally administrable, gelled oil-in-water emulsion as claimed in claim 12, wherein said bulking agents are present at a concentration of from 45 to 70 wt. %, preferably 50 to 65 wt. %, e.g. 55 to 60 wt. %, based on the aqueous phase.

14. An orally administrable, gelled oil-in-water emulsion as claimed in any one of the preceding claims having an oil phase which comprises one or more physiologically tolerable lipids derived from rapeseed oil, sunflower oil, corn oil, olive oil, sesame oil, palm kernel oil, coconut oil, a nut oil, algae oil or hemp oil.

15. An orally administrable, gelled oil-in-water emulsion as claimed in any one of the preceding claims having an oil phase which constitutes from 5 to 60 wt. %, preferably from 5 to 50 wt. %, more preferably from 10 to 45 wt. %, for example from 15 to 40 wt. %, from 15 to 30 wt. % or from 20 to 25 wt. %. of the emulsion.

16. An orally administrable, gelled oil-in-water emulsion as claimed in any one of the preceding claims which further comprises at least one pharmaceutically active agent.

17. An orally administrable, gelled oil-in-water emulsion as claimed in any one of the preceding claims which further comprises at least one nutraceutically active agent.

18. An orally administrable, gelled oil-in-water emulsion as claimed in claim 17, wherein said nutraceutically active agent is a vitamin or a mineral.

19. An orally administrable, gelled oil-in-water emulsion as claimed in any one of the preceding claims which is provided in unit dose form.

20. An orally administrable, gelled oil-in-water emulsion as claimed in claim 19, wherein said unit dose form is uncoated.

21. A package comprising an air-tight and light-tight compartment containing one dose unit of the gelled oil-in-water emulsion as claimed in claim 19 or claim 20.

22. A method for the preparation of an orally administrable, gelled oil-in-water emulsion as claimed in any one of claims 1 to 20, said method comprising the steps of: forming an oil phase which comprises one or more physiologically tolerable lipids; forming an aqueous phase comprising a gelling agent which is agar; combining said oil phase and said aqueous phase to form an oil-in-water emulsion in the presence of a surfactant which comprises an ester of glycerol in which at least one hydroxyl group is esterified by diacetyl tartaric acid and at least one hydroxyl group is esterified by a fatty acid; and allowing said emulsion to gel.

23. A gelled oil-in-water emulsion as claimed in any one of claims 1 to 20 for oral use as a medicament or for oral use in therapy.

24. A gelled oil-in-water emulsion as claimed in any one of claims 1 to 20 which contains at least one pharmaceutically active component for oral use in the treatment of a condition responsive to said pharmaceutically active component.

25. Use of a gelled oil-in-water emulsion as claimed in any one of claims 1 to 20 as a nutraceutical.
